(19) United States
(12) Patent Application Publication
(10) Pub. No.: US 2015/0037285 A1
(43) Pub. Date: Feb. 5, 2015

(54) METHODS FOR EFFICIENT TRANSFER OF VIABLE AND BIOACTIVE MICROBIOTA

(71) Applicant: NEW YORK UNIVERSITY, New York, NY (US)

(72) Inventors: Martin J. BLASER, New York, NY (US); Laura M. COX, Brooklyn, NY (US)

(73) Assignee: NEW YORK UNIVERSITY, New York, NY (US)

(21) Appl. No.: 14/323,760

(22) Filed: Jul. 3, 2014

Related U.S. Application Data
(60) Provisional application No. 61/842,893, filed on Jul. 3, 2013.

(51) Int. Cl. A61K 35/74; A61K 45/06

(52) U.S. Cl. CPC A61K 35/741; A61K 35/747; A61K 45/06; A61K 2035/115
USPC 424/93.3; 424/93.1; 424/93.45; 424/93.48

(57) ABSTRACT

The present invention relates to methods for transferring gastrointestinal microbiota that preserves viability and bioactivity of the microbiota, even if fastidious, anaerobic, and non-culturable organisms are present. Also provided herein are examples of how manipulating the gastrointestinal microbiota and introducing particular taxa can be used to affect host metabolic status related to weight, fat, and obesity.

### CROSS-REFERENCE TO RELATED APPLICATIONS

This application claims priority to U.S. Provisional Application Ser. No. 61/842,893, filed Jul. 3, 2013, which is herein incorporated by reference in its entirety.

### STATEMENT REGARDING FEDERALLY SPONSORED RESEARCH

Research and development leading to certain aspects of the present invention were supported, in part, by grants 1UL1RR029893 and R01DK090989 from the National Center for Research Resources, National Institutes of Health. Accordingly, the U.S. government may have certain rights in the invention.

### FIELD OF THE INVENTION

The present invention relates to methods for transferring gastrointestinal microbiota that preserves viability and bioactivity of the microbiota, even if fastidious, anaerobic, and non-culturable organisms are present. Also provided herein are examples of how manipulating the gastrointestinal microbiota and introducing particular taxa can be used to affect host metabolic status related to weight, fat, and obesity.

### BACKGROUND OF THE INVENTION

The intestinal microbiota is a diverse community composed of trillions of microbes that can either contribute to disease or promote health. The microbiota carry out essential functions such as vitamin synthesis, pathogen displacement, and aid in the development of the immune system.¹ It is critical for health to maintain a stable microbiota that is both resilient (able to recover from change) and resistant to invasion. Maintaining high diversity promotes stability, however, various insults impact the diversity in the gut. Antibiotics can limit the diversity within the gut, as well as diseases with high inflammation, such as inflammatory bowel disease (IBD).⁴ A healthy microbiota can protect against pathogen invasion, however, after a disturbance, as seen with antibiotic treatment, pathogenic organisms like Clostridium difficile can invade and cause disease. Infusion of microbiota from a healthy donor restores the pathogen barrier function and ameliorates Clostridium difficile associated diarrhea (CDI)². Microbiota transfers have also improved symptoms of IBD, irritable bowel syndrome (IBS), and idiopathic constipation.³

The current clinical methodology does not take measures to exclude oxygen, a critical step to preserve the viability of the anaerobic bacteria, which comprise the majority of the intestinal microbiota. The efficacy of microbiota transplants is variable, and can require more than one infusion.² One potential source for failure is the loss of viability of microorganisms in the donor sample. One study found 81% improvement in recurrent Clostridium difficile infection (rCDI) after one transplant and 94% improvement after the 2^{nd} transplant. With each procedure, there is risk and cost associated, and improving the efficiency of the initial transfer would reduce costs and patient discomfort. Other studies have similarly reported that fecal microbiota transplant fails in at least 1 of 10 cases.³

### SUMMARY OF THE INVENTION

As specified in the Background section above, there is a great need in the art for improving efficacy of microbiota transplants.

The present invention addresses these and other needs by providing a method for transferring microbiota that preserves viability and bioactivity of the microbiota, even if fastidious, anaerobic, and non-culturable organisms are present.

In one aspect, the invention provides a method for transfer of gastrointestinal microbiota from a donor subject to a recipient subject comprising the steps of:

(a) specimen collection, wherein a microbiota sample is recovered from the donor subject and, within 10 minutes of collection, is placed in an airtight collection container with or without an anaerobic transport medium, and sealed to avoid contact with oxygen in the air;
(b) specimen preparation, wherein the microbiota sample collected in step (a) is prepared in an anaerobic environment, comprising (i) adding a reduced (no oxygen) sterile solution if the microbiota sample was not collected in solution in step (a) or optionally adding a reduced (no oxygen) sterile solution if the microbiota sample was collected in solution in step (a), followed by (ii) homogenization, (iii) removal of solids, and (iv) transfer to a transport container that is under an anaerobic environment and has an airtight cap;
(c) transport of the microbiota sample prepared in step (b) to the delivery site in the recipient subject in the transport container;
(d) removal of the microbiota from the transport container into a delivery vehicle with minimal oxygen exposure, and
(e) direct transfer of the microbiota to the gastrointestinal tract of the recipient subject using the delivery vehicle, with minimal oxygen exposure.

In one embodiment, in step (a) the microbiota sample is recovered from the donor subject by recovery of feces immediately after defecation or by removal of cecal, ileal, or colonic luminal contents.

In one embodiment, in the collection step (a), the microbiota sample is placed in an airtight container within 1 minute of collection.

In one embodiment, the transport medium is step (a) is a reduced (no oxygen) sterile solution (e.g., saline, water, or other anaerobic transport media).

In one embodiment, the anaerobic environment in step (b) is composed of 90% nitrogen, 5% hydrogen, and 5% carbon dioxide. In another embodiment, the anaerobic environment in step (b) is composed of 95% nitrogen and 5% hydrogen. In yet another embodiment, the anaerobic environment in step (b) is composed of 100% nitrogen.

In one embodiment, the sterile solution in step (b) is selected from the group consisting of saline, water, milk, and other reduced solutions.

In one embodiment, step (a) and/or (b) is followed by freezing the microbiota sample and thawing said sample before the next step. In one specific embodiment, the microbiota is transferred to the recipient subject within 1 hour from the time of thawing of the frozen microbiota sample. In another specific embodiment, in step (c), transport is conducted for up to 4 hours from the time of thawing of the frozen microbiota sample.

In one embodiment, step (c) is conducted at room temperature or at 18-25° C.

In one embodiment, in step (c), transport is conducted for up to 4 hours after the specimen preparation of step (b).

In one embodiment, step (d) is conducted without opening the transport container with the microbiota sample using a needle (≦16 gauge) and syringe to pierce the airtight cap and draw up a sufficient volume of the microbiota suspension. In another embodiment, step (d) is conducted by transferring the microbiota suspension to a delivery vehicle (e.g., nasogastric tube, enema, capsule, or colonoscopy) within 3 minutes of opening the container with the microbiota sample.

In one specific embodiment, step (e) is accomplished by replacing the needle with a delivery vehicle (e.g., nasogastric tube, enema, capsule, or colonoscopy) that allows direct placement of the microbiota suspension in the gastrointestinal tract of the recipient subject.

In one embodiment, the microbiota is transferred to the recipient subject within 1 hour of inoculum preparation.

In one embodiment, the method of the invention preserves all major microbiota taxa, originating at levels >1% of the inoculum. In one embodiment, the method of the invention preserves at least 80% of the microbiota taxa originating at levels >0.1% of the inoculum. In one embodiment, the method of the invention preserves at least 70% of the microbiota taxa originating at levels >0.01% of the inoculum. In one embodiment, the method of the invention preserves more than 90% of the representation of the taxonomic abundances from the inoculum in the recipient subject.

In one embodiment, the method of the invention permits transfer of microbiota that modifies the recipient subject's metabolic status. In one embodiment, the method of the invention permits transfer of microbiota that modifies the recipient subject's immunological status.

In a related aspect, the invention provides a method for treating a disease in a subject in need thereof, wherein the disease is selected from the group consisting of Clostridium difficile associated diarrhea (CDI), inflammatory bowel disease (IBD), irritable bowel syndrome (IBS), idiopathic constipation, celiac disease, short stature, and growth retardation, said method comprising administering to the subject a therapeutically effective amount of a fecal microbiota transplant in accordance with the above transfer method of the invention.

In a separate aspect, the invention provides a method of treating or preventing weight gain and adiposity in a subject comprising administering to the subject a therapeutically effective amount of a microbiota inoculum comprising bacteria from one or more of the following taxa: order Mollicutes order RF39, order Lactobacillales, family Coriobacteriaceae, family Rikenellaceae, family Clostridiaceae, family Peptostreptococcaceae, family Lactobacillaceae, genus Allobaculum, genus Klebsiella, genus Ruminococcus, genus Dorea, genus Lactobacillus, genus Peptococcaceae genus rc4-4, genus Desulfovibrio, genus Clostridiaceae genus SMB53, genus Roseburia, genus Oscillospira, species Lactobacillus reuteri.

In another separate aspect, the invention provides a method of promoting and/or enhancing weight gain and/or height gain and/or fat accumulation in a subject in need thereof comprising administering to the subject a therapeutically effective amount of a microbiota inoculum comprising bacteria from one or more of the following taxa: family Verrucomicrobiaceae, family Lachnospiraceae, family Porphyromonadaceae, family Enterococcaceae, genus Akkermansia, genus Odoribacter, genus Enterococcus, genus Blautia, species Akkermansia muciniphila, species Blautia producta.

In one embodiment of each of the above methods, the method comprises the above microbiota transfer method of the invention.

In one embodiment of each of the above methods, the method further comprises administering a prebiotic or a probiotic to promote growth and/or activity of the relevant taxa.

In another separate aspect, the invention provides a method for predicting an increase in weight, height, and adiposity in a subject, said method comprising detecting in the gastrointestinal microbiota of the subject one or more bacterial taxa selected from the group consisting of family Verrucomicrobiaceae, family Lachnospiraceae, family Porphyromonadaceae, family Enterococcaceae, genus Akkermansia, genus Odoribacter, genus Enterococcus, genus Blautia, species Akkermansia muciniphila, and species Blautia producta.

In another aspect, the invention provides a method for predicting a decrease in weight, height, and adiposity in a subject, said method comprising detecting in the gastrointestinal microbiota of the subject one or more bacterial taxa selected from the group consisting of order Mollicutes order RF39, order Lactobacillales, family Coriobacteriaceae, family Rikenellaceae, family Clostridiaceae, family Peptostreptococcaceae, family Lactobacillaceae, genus Allobaculum, genus Klebsiella, genus Ruminococcus, genus Dorea, genus Lactobacillus, genus Peptococcaceae genus rc4-4, genus Desulfovibrio, genus Clostridiaceae genus SMB53, genus Roseburia, genus Oscillospira, and species Lactobacillus reuteri.

In one embodiment of the above two methods, bacterial taxa are identified by high-throughput 16S rRNA sequencing.

In one embodiment of any of the above methods, the subject is human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of the method of the invention, including collection, sample preparation, and transfer of living microbiota.

FIG. 2 shows the study design for transmission of altered host phenotypes through microbiota transfer. C57BL6J mice either did not receive antibiotics (Control) or received sub-therapeutic antibiotic treatment (STAT) (penicillin) from birth until 18 weeks of age. Mice were fed normal chow, then switched to high fat diet at 6 weeks of age. At 18 weeks, cecal contents were collected from 3 control and 3 STAT mice, based on their median weight, pooled, and transferred to 3-4 week old germ-free Swiss Webster mice by oral gavage. By continuing strict anaerobiosis and reducing conditions, every attempt was made to maintain viability of the microbiota by protecting the microbiota from oxygen and minimizing time and exposure of ex vivo microbiota collection and transfer (see Example 1). Microbiota recipient mice were given a high fat diet and monitored for 5 weeks. Longitudinal fecal samples were collected to assess the efficiency of the microbiota transfer.

FIGS. 3A-G show microbiota transfer efficiency from control mice to germ-free mice. The cecal microbiota was collected from 3 conventional C57BL6 mice in anaerobic transport media, mixed in reduced (no oxygen), sterile saline in an anaerobic chamber, and transferred to 7 germ-free Swiss Webster mice. The donor cecal samples, the inoculum, and recipient intestinal microbiota were assessed by 16S rRNA high-throughput sequencing. (A) Rank abundance plot of the 72 bacterial species detected in the inoculum, stratified by relative abundance: high (>1%), mid (0.1-1%), low (0.01-0.1%), and only detected from a single sequence. The inoculum was sequenced at a depth of 11,171 reads. (B) Transmission of the 72 species from the inoculum to the 7 germ-free recipient mice, stratified by the relative abundance of each species in the inoculum. (C) Scatter plot of species occurrence in the 7 recipient mice stratified by abundance in inoculum, or detected in the individual donor cecal samples but not pooled inoculum, or new species not detected in the donor or inoculum samples. (D) Inoculum species occurrence in the 69 recipient samples. (E) Transfer efficiency of the 72 inoculum species over time. (F) The overall contribution to relative abundance of high, mid, low, and very low abundance microbiota in the individual donor cecal samples and inoculum. (G) The proportion (relative abundance) of microbiota in the recipient mice from the inoculum, the individual donor specimens, or new species. (Plots B-G show mean±standard error).

FIGS. 4A-G show microbiota transfer efficiency with a manipulated-microbiota donor source to germ-free mice. 3 conventional C57BL6 mice received STAT for 18 weeks, then cecal microbiota was collected in anaerobic transport media, mixed in reduced (no oxygen), sterile saline in an anaerobic chamber, and transferred to 8 germ-free Swiss-Webster mice. The donor cecal samples, the inoculum, and recipient intestinal microbiota was assessed by 16S rRNA high-throughput sequencing. (A) Rank abundance plot of the 50 bacterial species detected in the inoculum, stratified by relative abundance: high (>1%), mid (0.1-1%), low (<0.1%, greater than 1 sequence), and only detected from a single sequence. The inoculum was sequenced at a depth of 6,641 reads. (B) Transmission of the 50 species from the inoculum to the 8 germ-free recipient mice, stratified by the relative abundance of each species in the inoculum. (C) Scatter plot of species occurrence in the 8 recipient mice stratified by abundance in inoculum, or detected in the individual donor cecal samples but not the pooled inoculum, or new species not detected in the donor or inoculum samples. (D) Inoculum species occurrence in the 79 recipient samples. (E) Transfer efficiency of the 50 inoculum species over time. (F) The overall contribution to relative abundance of high, mid, low, and very low abundance microbiota in the donor cecal samples and inoculum. (G) The proportion (relative abundance) of microbiota in the recipient mice from the inoculum, the individual donor specimens, or new species. (Plots B-G show mean±standard error).

FIGS. 5A-B show distribution of inoculum species transmissibility. Histogram of species transmission into recipient mice for species with high abundance, mid abundance, low abundance, or detected by a single read in the inoculum, or detected in the individual donor specimens but not in the pooled inoculum, or new: not detected in the donor or inoculum samples. Panel A: control microbiota recipients, Panel B: STAT-microbiota recipients.

FIGS. 6A-G show microbiota transfer efficiency from conventionalized (control) formerly germ-free mice to 6 new germ-free mice of the same strain. Germ-free Swiss Webster mice were colonized with control-microbiota at 3 weeks of age, then, these now conventionalized mice were sacrificed at 8 weeks of age. Cecal microbiota was collected from 3 colonized Swiss-Webster mice in anaerobic transport media, mixed in reduced (no oxygen), sterile saline in an anaerobic chamber, and transferred to 6 new germ-free Swiss Webster mice. The donor cecal samples, the inoculum, and recipient intestinal microbiota was assessed by 16S rRNA high-throughput sequencing. (A) Rank abundance plot of the 71 bacterial species detected in the inoculum, stratified by relative abundance: high (>1%), mid (0.1-1%), low (<0.1%, greater than 1 sequence), and only detected from a single sequence. The inoculum was sequenced at a depth of 13,151 reads. (B) Transmission of the 71 species from the inoculum to the 6 germ-free recipient mice, stratified by the relative abundance of each species in the inoculum. (C) Scatter plot of species occurrence in the 6 recipient mice stratified by abundance in inoculum, or detected in the individual donor cecal samples but not pooled inoculum, or new species not detected in the donor or inoculum samples. (D) Inoculum species occurrence in the 23 recipient samples. (E) Transfer efficiency of the 71 inoculum species over time. (F) The overall contribution to relative abundance of high, mid, low, and very low abundance microbiota in the donor cecal samples and inoculum. (G) The proportion (relative abundance) of microbiota in the recipient mice from the inoculum, the individual donor specimens, or new species. (Plots B-G show mean±standard error).

FIGS. 7A-G show microbiota transfer efficiency from conventionalized (STAT) formerly germ-free mice to germ-free mice. Germ-free Swiss Webster mice were colonized with STAT-microbiota at 3 weeks of age, then sacrificed at 8 weeks of age. Cecal microbiota was collected from 3 colonized Swiss-Webster mice in anaerobic transport media, mixed in reduced (no oxygen), sterile saline in an anaerobic chamber, and transferred to 6 new germ-free Swiss Webster mice. The donor cecal samples, the inoculum, and recipient intestinal microbiota was assessed by 16S rRNA high-throughput sequencing. (A) Rank abundance plot of the 70 bacterial species detected in the inoculum, stratified by relative abundance: high (>1%), mid (0.1-1%), low (<0.1%, greater than 1 sequence), and only detected from a single sequence. The inoculum was sequenced at a depth of 11,423 reads. (B) Transmission of the 70 species from the inoculum to the 6 germ-free recipient mice, stratified by the relative abundance of each species in the inoculum. (C) Scatter plot of species occurrence in the 6 recipient mice stratified by abundance in inoculum, or detected in the individual donor cecal samples but not the pooled inoculum, or new species not detected in the donor or inoculum samples. (D) Inoculum species occurrence in the 24 recipient samples. (E) Transfer efficiency of the 70 inoculum species over time. (F) The overall contribution to relative abundance of high, mid, low, and very low abundance microbiota in the donor cecal samples and inoculum. (G) The proportion (relative abundance) of microbiota in the recipient mice from the inoculum, the individual donor specimens, or new species. (Plots B-G show mean±standard error).

FIGS. 8A-B show distribution of inoculum species transmissibility. Histogram of species transmission into recipient mice for species with high abundance, mid abundance, low abundance, or detected by a single read in the inoculum, or detected in the individual donor specimens but not in the pooled inoculum, or new: not detected in the donor or inoculum samples. Panel A: control microbiota recipients, Panel B: STAT-microbiota recipients.

FIGS. 9A-B show depth of coverage in microbiome transfer and recipient samples. (A) Number of 16S rRNA microbial sequences surveyed in the individual donor samples (n=3), the pooled inoculum (n=1), and the recipient fecal, cecal, and ileal samples in control (CT1, n=7), and STAT (ST1, n=8) germ-free microbiota-recipients. (A) Number of 16S rRNA microbial sequences surveyed in the individual donor samples (n=3, coming from CT1 or ST1 mice), the pooled inoculum (n=1), and the recipient fecal, cecal, and ileal samples in control (CT2, n=6), and STAT (ST2, n=6) germ-free microbiota-recipients.

FIGS. 10A-I show metabolic and ecological consequences of transferring STAT microbiota. Cecal microbiota from 3 control and 3 STAT C57B/L6J mice at 18 weeks of age were collected, pooled in a saline solution, and transferred to 3-week old germ-free Swiss-Webster mice by oral gavage. (A) Microbiota donors were selected based on the median total mass determined by DEXA scanning at 16-weeks. (B) Scale weight of recipient mice. (C) Total, lean, and fat mass in conventionalized germ-free recipient mice over 35 days determined by DEXA scanning. There were significant (p<0.05, t-test) increases in the total mass and fat mass of the mice receiving the cecal microbiota from the donor mice that had received the STAT penicillin. (D-F) Community structure assessed by PCoA of unweighted UniFrac distances of the donor cecal, the transferred inoculum, and the recipient mouse fecal samples at 1 (D), 9 (E), and 34 (F) days post-transfer, colored by sample type: donor cecum; the transferred inoculum; and the recipient mouse fecal, cecal, and ileal samples. The 3 axes account for 24.3% of the total variation. (G) Mean unweighted UniFrac distance from inoculum, * p<0.05 t-test. (H) α-diversity in donors, inoculum, and recipients calculated at an even sampling depth of 1170. (I) Relative abundance at the class levels in the donor, the transferred inoculum, and the recipient mice over time. The height of each color corresponds to the population levels (%). The taxa displayed had a maximum relative abundance >2% at any time point within a group.

FIG. 11 shows microbial correlations with fat mass. Germ-free Swiss Webster mice were colonized with microbiota from Control or STAT mice. The intestinal microbiota of the recipients was surveyed over time (1-34-days post-transfer fecal specimens, cecal and ileal specimens 35-days post-transfer) by high throughput sequencing at an mean±SD depth of 6729±3334 sequences per sample. Taxonomic assignment used the QIIME pipeline based on the May 20, 2013 Green Genes database of 16S microbial sequences. The Spearman correlation was calculated with reference to fat mass at 34-days-post transfer with relative abundance of the predominant species (>1% in any sample). Microbiota with at least one significant correlation (p<0.05), and consistent correlation direction are shown. An ellipse with a forward slant represents a positive Spearman correlation, and a backwards slant represents a negative Spearman correlation, and the narrowness of the ellipse indicates the strength of the correlation (higher rho value). Microbiota are reported at the lowest possible identifiable level, indicated by the letter preceding the underscore: o=order, f=family, g=genus, s=species. This example defines the significant taxa to the genus level in most cases, and including the species level, and represents candidate microbiota for manipulating fat mass, extending the observations in Table 6.

### DETAILED DESCRIPTION OF THE INVENTION

The present inventors have hypothesized that current practices such as improper transport and storage of anaerobic organisms, or homogenization of the microbiota transplant specimens at ambient atmospheres (with oxygen), may be the cause of failed microbiota transplants. Microbiota transplants rely on bioactive and viable microorganisms. One means of improving the success rate is adequate preservation of the biological substances being transferred. Since the fecal microbiota transplants contain many steps that may occur over a long period of time (months or years, if donor microbiota specimens are frozen for future use), the present inventors have hypothesized that it is essential to exclude oxygen to the maximal extent in each step of the process: this will ensure that even if delays are present, the anaerobic microbiota will remain viable. The present inventors have thus developed a method for microbiota transplant, wherein microbiota is protected during transport from donor collection, during inoculum (infusion) preparation, and during transport to the recipient. Maintenance of microbiota viability in the method of the present invention is an essential factor when considering regulation of fecal microbiota transplants as a therapeutic intervention.

Studies have shown that in successful fecal transplant cases, the recipient microbiota resembles the healthy donor microbiota. Resolution of disease has been associated with increases in Clostrial Clusters IV and XIVa and Bacteroidetes, and decreases in Proteobacteria.³ The exclusion of oxygen during the fecal microbiota transplantation (FMT) in the method of the present invention increases the viability of Clostridial Clusters IV and XIVa and Bacteroidetes and improves the success rate of FMT.

### DEFINITIONS

As used herein, the term “bacteria” encompasses both prokaryotic organisms and archaea present in mammalian microbiota.

The terms “intestinal microbiota”, “gut flora”, and “gastrointestinal microbiota” are used interchangeably to refer to bacteria in the digestive tract.

Specific changes in microbiota discussed herein can be detected using various methods, including without limitation quantitative PCR or high-throughput sequencing methods which detect over- and under-represented genes in the total bacterial population (e.g., 454-sequencing for community analysis; screening of microbial 16S ribosomal RNAs (16S rRNA), etc.), or transcriptomic or proteomic studies that identify lost or gained microbial transcripts or proteins within total bacterial populations. See, e.g., U.S. Patent Publication No. 2010/0074872; Eckburg et al., Science, 2005, 308:1635-8; Costello et al., Science, 2009, 326:1694-7; Orrice et al., Science, 2009, 324:1190-2; Li et al., Nature, 2010, 464: 59-65; Bjursell et al., Journal of Biological Chemistry, 2006, 281:36269-36279; Mahowald et al., PNAS, 2009, 14:5859-5864; Wikoff et al., PNAS, 2009, 10:3698-3703.

As used herein, the term “probiotic” refers to a substantially pure bacteria (i.e., a single isolate, live or killed), or a mixture of desired bacteria, or bacterial extract, and may also include any additional components that can be administered to a mammal. Such compositions are also referred to herein as a “bacterial inoculant.” Probiotics or bacterial inoculant compositions of the invention are preferably administered with a buffering agent (e.g., to allow the bacteria to survive in the acidic environment of the stomach and to grow in the intestinal environment). Non-limiting examples of useful buffering agents include saline, sodium bicarbonate, milk, yogurt, infant formula, and other dairy products.

As used herein, the term “prebiotic” refers to an agent that increases the number and/or activity of one or more desired bacteria. Non-limiting examples of prebiotics useful in the methods of the present invention include fructooligosaccharides (e.g., oligofructose, inulin, inulin-type fructans), galactooligosaccharides, N-acetylglucosamine, N-acetylgalactosamine, glucose, other five- and six-carbon sugars (such as arabinose, maltose, lactose, sucrose, cellobiose, etc.), amino acids, alcohols, resistant starch (RS), and mixtures thereof. See, e.g., Ramirez-Farias et al., Br J Nutr (2008) 4:1-10; Pool-Zobel and Sauer, J Nutr (2007), 137:2580S-2584S.

As used herein, the term “metagenome” refers to genomic material obtained directly from a subject, instead of from culture. Metagenome is thus composed of microbial and host components.

The terms “treat” or “treatment” of a state, disorder or condition include:
(1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or
(2) inhibiting the state, disorder or condition, i.e., arresting, reducing or delaying the development of the disease or a relapse thereof (in case of maintenance treatment) or at least one clinical or sub-clinical symptom thereof; or
(3) relieving the disease, i.e., causing regression of the state, disorder or condition or at least one of its clinical or sub-clinical symptoms.

The benefit to a subject to be treated is either statistically significant or at least perceptible to the patient or to the physician.

As used herein in connection with administration of antibiotics, the term “antibiotic treatment” comprises antibiotic exposure.

As used herein, the term “early in life” refers to the period in life of a mammal before growth and development is complete. In case of humans, this term refers to pre-puberty, preferably within the first 6 years of life.

A “therapeutically effective amount” means the amount of a bacterial inoculant or a compound (e.g., an antibiotic or a prebiotic) that, when administered to a subject for treating a state, disorder or condition, is sufficient to effect such treatment. The “therapeutically effective amount” will vary depending on the compound, bacteria or analogue administered as well as the disease and its severity and the age, weight, physical condition and responsiveness of the mammal to be treated.

When used in connection with antibiotic administration, the term “therapeutic dose” refers to an amount of an antibiotic that will achieve blood and tissue levels corresponding to the minimal inhibitory concentration (MIC) for at least 50% of the targeted microbes, when used in a standardized in vitro assay of susceptibility (e.g., agar dilution MICs; see Manual of Clinical Microbiology, ASM Press).

The term “sub-therapeutic antibiotic treatment” or “sub-therapeutic antibiotic dose” refers to administration of an amount of an antibiotic that will achieve blood and tissue levels below the minimal inhibitory concentration (MIC) for 10% of targeted organisms, when used in a standardized in vitro assay of susceptibility (e.g., agar dilution MICs; see Manual of Clinical Microbiology, ASM Press). Non-limiting examples of useful doses for sub-therapeutic antibiotic treatment include 1-5 mg/kg/day.

As used herein, the phrase “pharmaceutically acceptable” refers to molecular entities and compositions that are generally regarded as physiologically tolerable.

As used herein, the term “combination” of a bacterial inoculant, probiotic, analogue, or prebiotic compound and at least a second pharmaceutically active ingredient means at least two, but any desired combination of compounds can be delivered simultaneously or sequentially (e.g., within a 24 hour period).

“Patient” or “subject” as used herein refers to mammals and includes, without limitation, human and veterinary animals.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

### Method of the Invention

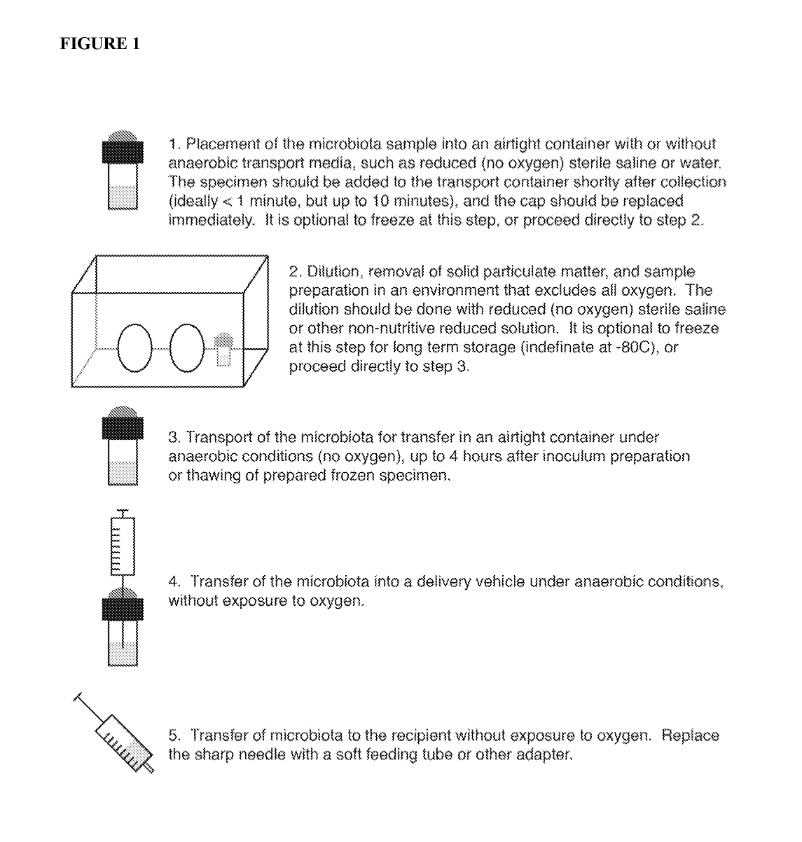

The steps of the method of the invention are summarized in FIG. 1 and are also described below.

### 1. Specimen Collection

Recover microbiota and within 10 minutes of collection, place in a container with or without anaerobic transport medium (such as, e.g., reduced (no oxygen) saline or water), seal with an airtight cap. If administering to the microbiota recipient on another day, it is possible to freeze and save at this point (e.g., at −80° C.) for an indefinite length of time (years). It is essential the microbiota is contained in an airtight container, with various options for the type of container.

### 2. Specimen Preparation

Prepare the sample in an anaerobic environment (typically composed of 90% nitrogen, 5% hydrogen, and 5% carbon dioxide, or alternately 95% nitrogen, 5% hydrogen, or 100% nitrogen). It is essential that the environment excludes oxygen. Add pre-reduced anaerobically sterilized saline or other diluent (such as, e.g., water or milk), homogenize using a vortex, remove solids, and transfer to an airtight container with a Hungate cap (plastic cap with an airtight rubber septum). If administering to the microbiota recipient on another day, it is possible to freeze and save at this point (e.g., at −80° C.) for years.

3. Transport from the Preparation Site to the Delivery Site

Transport the microbiota specimen to the site of delivery in a container that is under an anaerobic environment and has an airtight cap. Room temperature (18-25° C.) is sufficient for this step, but not critical. It is essential that the container be airtight to exclude oxygen.

4. Removal from the Transport Container into the Delivery Vehicle

Method A: Without opening the container with the microbiota sample, use a needle (≦16 gauge) and syringe to pierce the rubber septum, draw up a sufficient volume of microbiota/saline mixture. Method B: Rapidly (<3 minutes) transfer the microbiota suspension to the transfer device (e.g., nasogastric tube, enema, capsule). Oxygen exposure for a short duration (<2 minutes) is acceptable when transferring the donor microbiota solution to the recipient. It is optimal to exclude oxygen at this step but not essential.

5. Direct transfer of the microbiota to the gastrointestinal tract.

Ideally, the microbiota should be transferred to the recipient within 1 hour of inoculum preparation or from the time of thawing the frozen prepared specimen. Replace the sharp needle with a feeding tube or other attachment that will allow direct placement of the microbiota/saline suspension in the gastrointestinal tract of the microbiota recipient. The donor microbiota can be transferred to the recipient, e.g., by nasogastric tube, enema, orcolonoscopy.

In accordance with the present invention there may be numerous tools and techniques within the skill of the art, such as those commonly used in molecular immunology, cellular immunology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Transmission of Normal or Manipulated Microbiota with High Efficiency and Viability of the Microbiota

C57BL/6J (Jackson Labs, Bar Harbor Me.) mice either received no antibiotics (control) or continuous subtherapeutic antibiotic treatment (STAT) with penicillin in their drinking water. Mice were weaned at 4 weeks onto normal chow (13.2% fat, 5053 PicoLab Rodent Diet 20, LabDiet, Brentwood, Mo.) then changed to a high fat diet (45% kcal from fat, D12451, Research Diets, New Brunswick, N.J.) at 6 weeks of life. At 18 weeks of age, the three animals with weight at or closest to the median were selected as cecal content donors from each group (Control, n=7; STAT, n=8). Donor mice were humanely euthanized, and the proximal ⅓ of the cecum was aseptically removed and immediately (less than 1 minute) placed in reduced (no oxygen), sterile liquid dental transport media (Anaerobe Systems, Morgan Hill Calif.). The cecal samples in anaerobic transport media were brought into an anaerobic chamber within an hour of collection (Sheldon, Cornelius Oreg.). The three cecal samples from each group (STAT or Control) were pooled, and reduced, sterile saline was added to a final volume of 8 mL, of which 3 mL was used for microbiota transfer. To maintain viability of anaerobic organisms, vials containing the inoculum were not opened. Instead the suspension was drawn through a rubber Hungate cap using a sharp needle, which was then replaced with a soft 20-gauge feeding tube (Fisher Science, Pittsburgh Pa.) for the oral gavage. Then, 3- to 4-week old germ-free Swiss Webster mice (Taconic Farms, Germantown N.Y.) were anesthetized using isoflurane, and 250 μL of either the pooled microbiota suspensions were placed in the stomachs of the germ-free Swiss-Webster mice by oral gavage (control microbiota recipients, n=7 recipients; STAT-microbiota recipients, n=8 recipients). Recipients were chosen randomly, and the inoculation procedure alternated between control and STAT recipients. Gloves were changed between every inoculation. Mice awoke from anesthesia within minutes and no mouse exhibited ill effects from the microbiota transfer. The microbiota-recipient mice were housed in autoclaved cages, under specific pathogen-free conditions, and fed an irradiated high fat diet (45% kcal from fat, D12451, Research Diets, New Brunswick, N.J.), and followed for the next 35 days until sacrifice. Fecal pellets were collected serially from the time of transfer, and cecal and ileal contents obtained at sacrifice for examined to assess transfer efficiency.

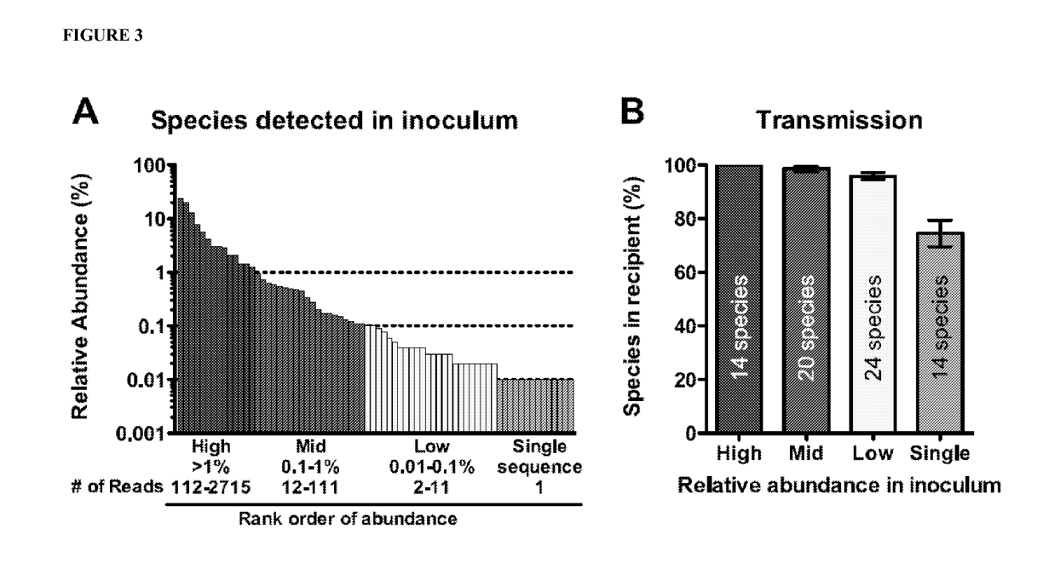

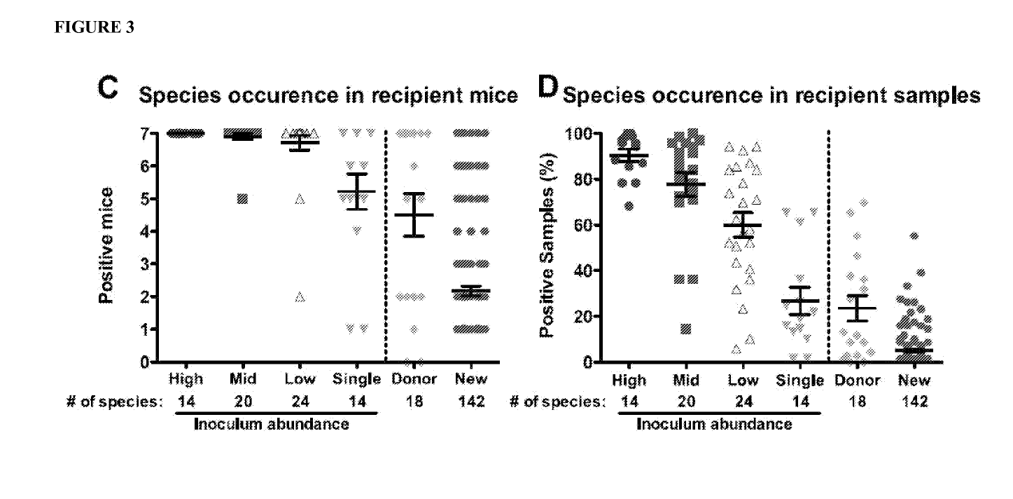

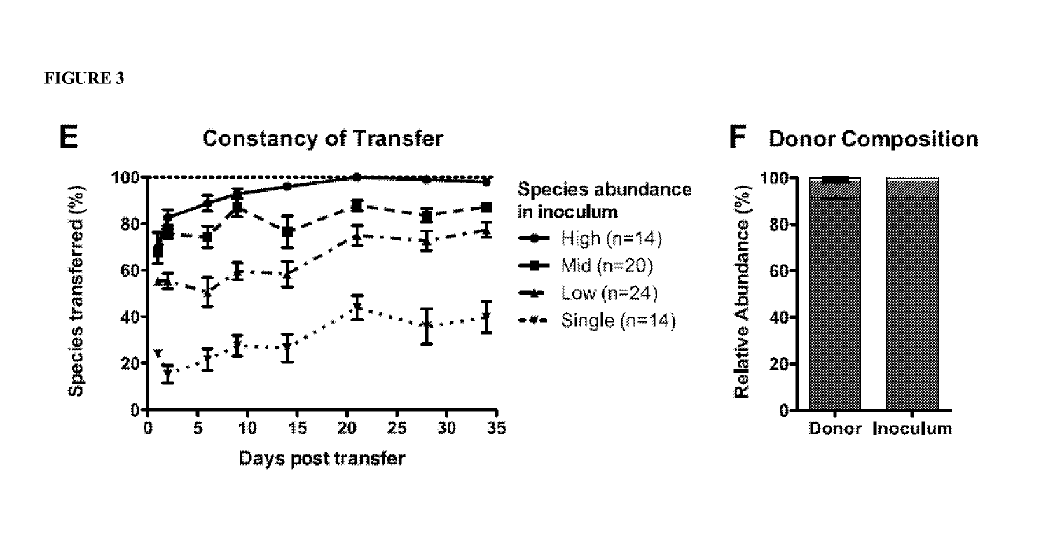

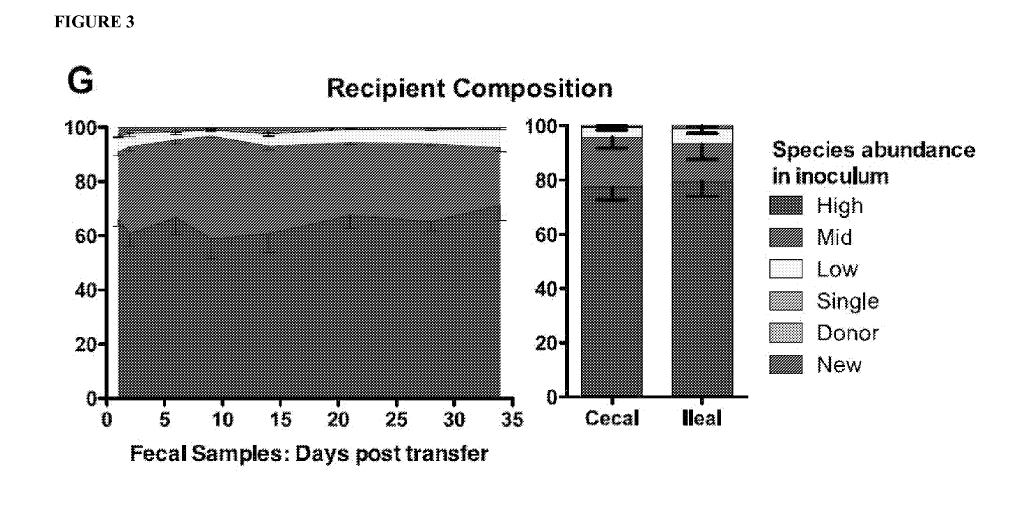

The control inoculum had a total of 72 species detected in a sample of 11,171 sequences of 16S rRNA. Of the 72 species, there were 14 species with high abundance (>1%), 20 species with moderate abundance (0.1-1%), 24 species with low abundance (0.01-0.1%), and 14 species that were detected by only a single read, which may be due to sequencing artifacts or true biological representation (FIG. 3A). All of the species with high abundance in the inoculum were detected in all control recipient mice (n=7), making the transfer efficiency 100% (FIG. 3B). These top 14 species accounted for 91.6% of the inoculum microbiota (FIG. 3F) and their populations decreased but remained dominant in the microbiota of the recipient mice, accounting for 67.5% of the recipient microbiota (FIG. 3G). Of the 20 moderately abundant species, there was an average transfer efficiency of 98.6±2.4% (3B), 19 of the 20 species were transferred to all 7 mice, while Anaeroplasma was transferred to 5 of 7 mice (3C). The 20 moderately abundant species increased their representation (7.3% of the inoculum composition to 26.4% of the recipient composition, FIGS. 3F-G), but were still less than the highly abundant organisms, thus the overall population patterns were conserved in the new hosts. Akkermansia mucinophila increased from 0.5% in the inoculum to 17.4% in the recipient mice, accounting for most of the difference. The moderately abundant and highly abundant species from the inoculum represented 93.9% of the recipient microbiota. The 24 species with low abundance (0.01%-0.1% of the inoculum) had an average transfer efficiency of 95.8±3.4% (3B). 22 of the 24 species were transferred to all 7 recipients, a member of the family Desulfovibrionaceae was detected in 5 out of 7 mice and an unclassified member of the Clostridiaceae family was detected in 2 mice. The 24 species with low abundance in the inoculum accounted for 0.98% of the inoculum microbiota and 4.6% of the recipient microbiota (2C). There were 14 species with extremely low abundance (<0.01% of the inoculum, 1 sequence detected), which had a transfer efficiency of 74.5±12.9%, and accounted for 0.13% of the inoculum microbiota and 0.3% of the recipient mouse microbiota. The recipient mice had 8 fecal samples, and 1 cecal and ileal sample. Allobaculum, a member of the Bacteroidales order, family S24-7, and a member of the Clostridiales family were detected in all samples (Table 5). Other species had lower detection levels in the recipient samples (FIG. 3D, Table 5). The lowest detection of inoculum species in the recipient was at 1-day post transfer, which increased overtime, likely reflecting a lag period where some transferred microbiota needed time to actively grow to detectable levels. This shows that the microbial community is reassembling in a new host, a characteristic of resilience, rather than drifting away from the founder community.

These data indicate that the microbiota was transferred effectively, with high recovery of the original organisms, and with maintenance of existing community structure. Low abundance species that were only detected in the individual donor cecal samples, but not in the inoculum due to the probability of detecting at the current sequencing depth, were detected in the recipient mice, accounting for 0.3±0.2% of the recipient microbiota. There were some new species detected in the recipient microbiota, but they only accounted for 0.9±0.7% of the microbiota, indicating that the microbiota in the inoculum were able to be successfully transferred, colonize, and develop stable populations that are resistant to invasion. The transfer maintained viability of the microbiota. Species with lower abundance in the inoculum had lower detection rates in recipient mice. Lack of finding these organisms in the recipient fecal pellets may reflect their loss (and non-transfer), or they may be present but not detected at the depth of sequencing. The gradual increases may represent the growth of the organisms to at least the level of sequencing detection (average 6729±3334 SD reads/sample).

### Example 2

### Transtat: Transmission of Altered Metabolic Phenotype Through Microbiota Transfer

The present inventors found strong associations between the receipt of sub-therapeutic antibiotic treatment (STAT) (penicillin) and changes in body composition in comparison to the mice that received Control drinking water. These observations suggest that the antibiotic exposure led to the changes in body composition, since it was the only variable in the experiment. However, to develop practical approaches to the causation of obesity, it is important to determine whether the antibiotics are working directly on the tissues or whether the effect of the antibiotic is mediated through its effects on microbiome composition. Therefore, the present inventors undertook an experiment to harvest microbiota from the STAT-exposed mice and the Control mice, and transfer them into germ-free mice. These mice now were conventionalized (i.e., they now were colonized by a microbiota), and the present inventors sought to determine the effects of the alternate sources of their microbiota on their immune characteristics. Transfer of microbiota to germ-free animals is now an accepted procedure to examine the characteristics of the microbiota, independent of any on-going host or drug effects.

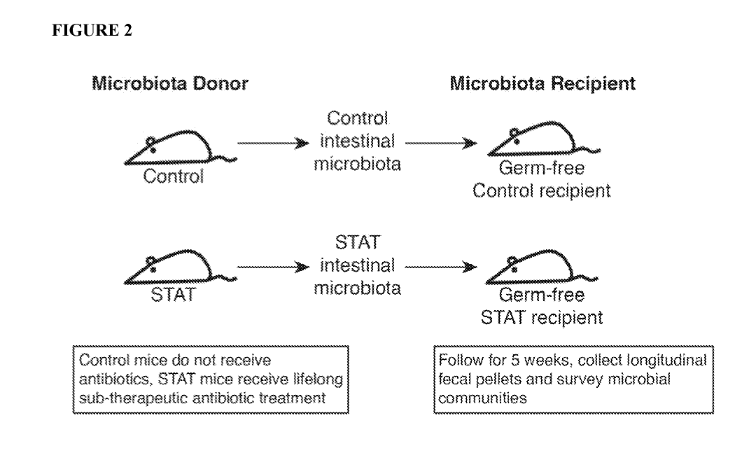

FIG. 2 shows a study design for transmission of altered host phenotypes through microbiota transfer. C57BL6J mice either did not receive antibiotics (Control) or received sub-therapeutic antibiotic treatment (STAT) penicillin (1 mg/kg body weight) from birth until 18 weeks of age. Mice were fed normal chow, then switched to high fat diet at 6 weeks of age. At 18 weeks, cecal contents were collected from 3 control and 3 STAT mice, based on their median weight, pooled, and transferred to 3-4 week old germ-free Swiss Webster mice by oral gavage. By continuing strict anaerobiosis and reducing conditions, every attempt was made to maintain viability of the microbiota by protecting the microbiota from oxygen and minimizing time and exposure of ex vivo microbiota collection and transfer (see Example 1). Microbiota recipient mice were given a high fat diet and monitored for 5 weeks. Longitudinal fecal samples were collected to assess the efficiency of the microbiota transfer.

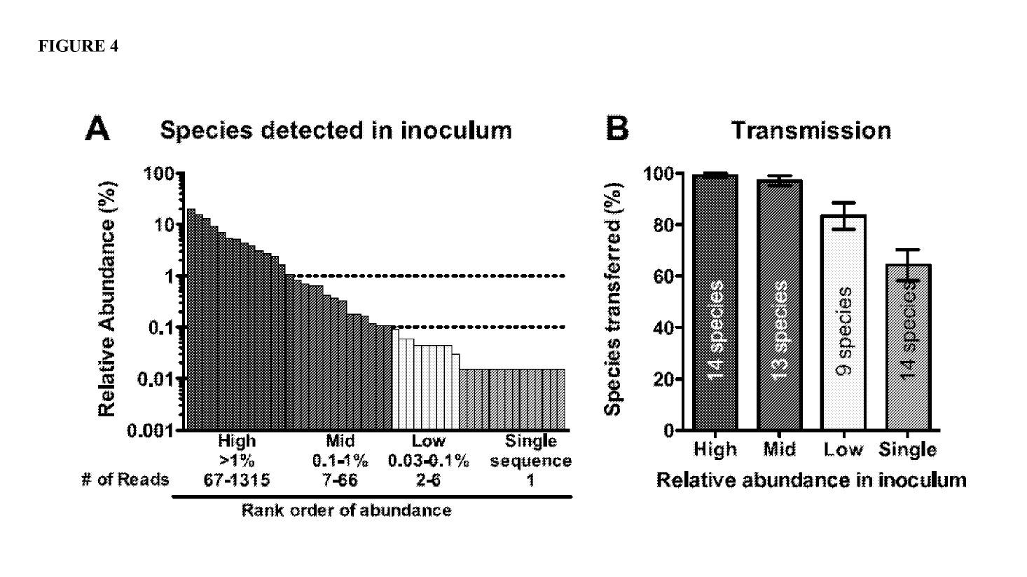

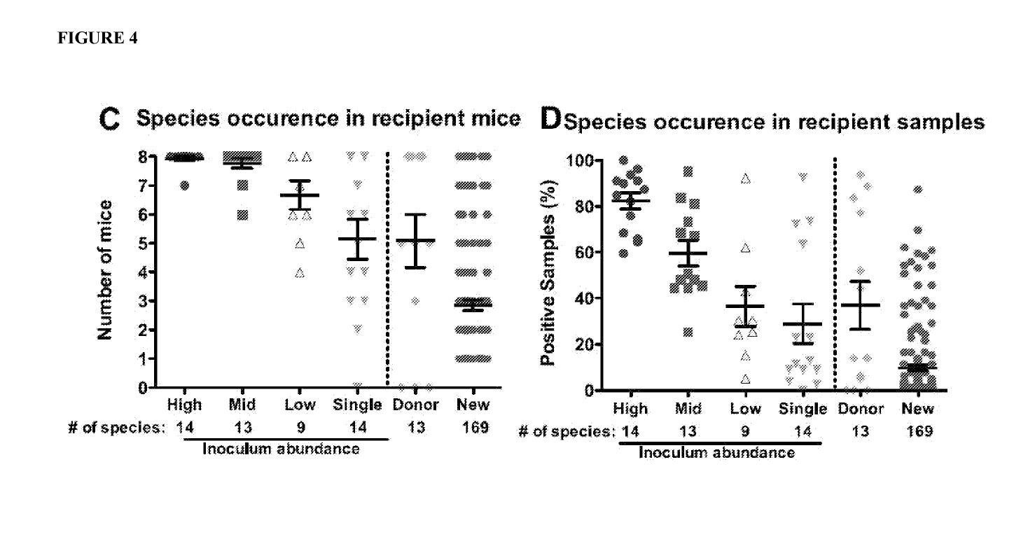

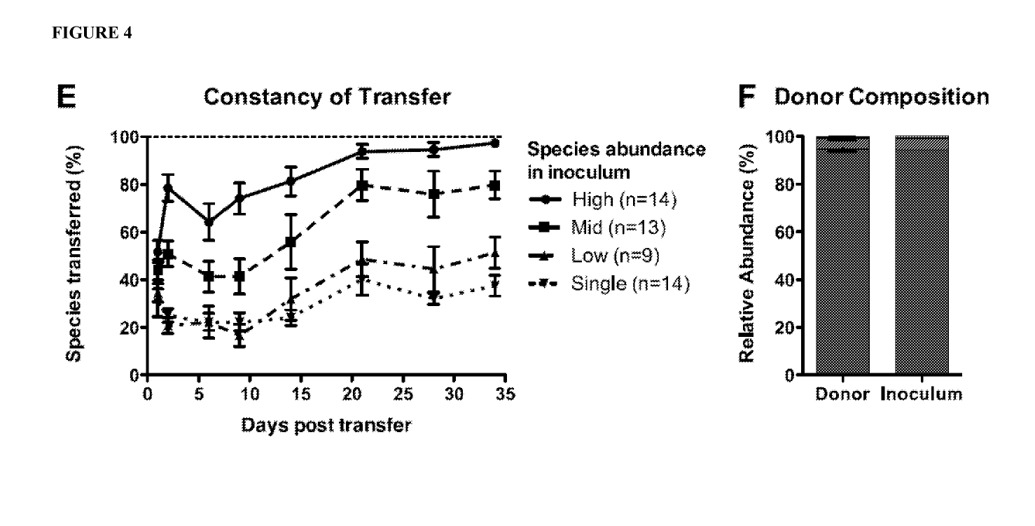

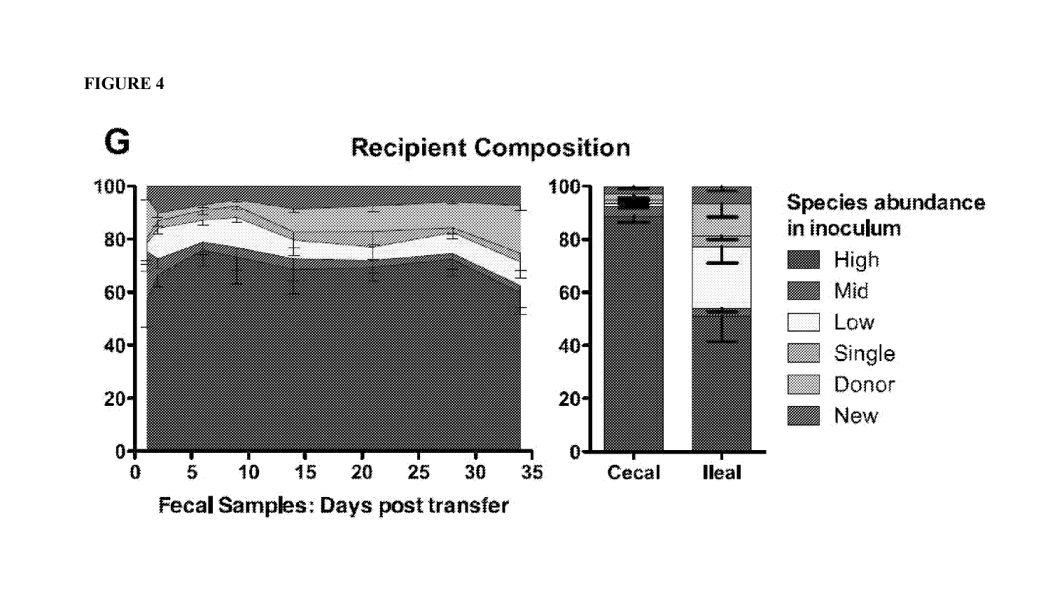

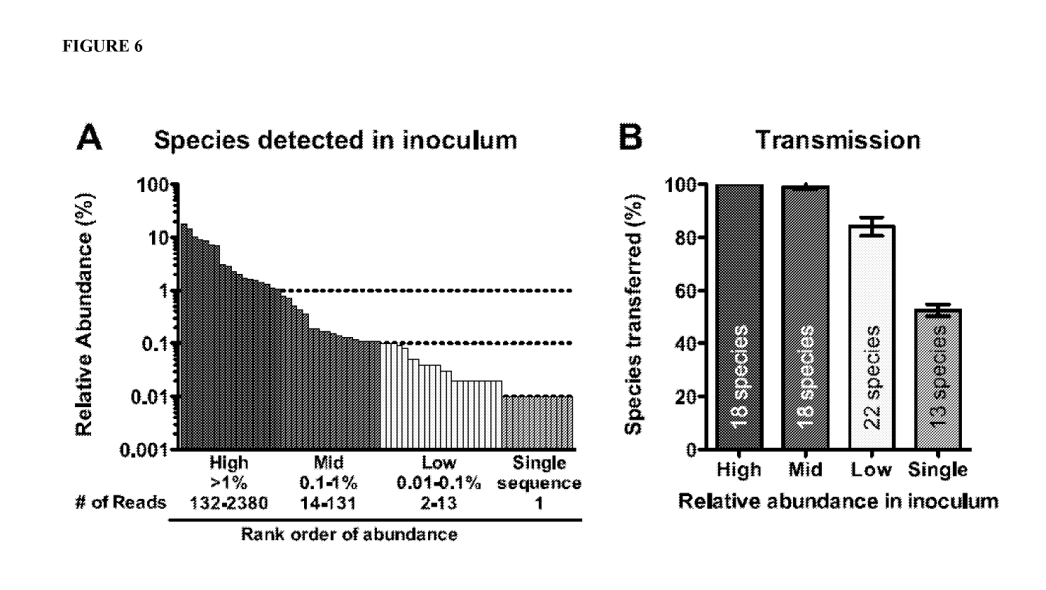

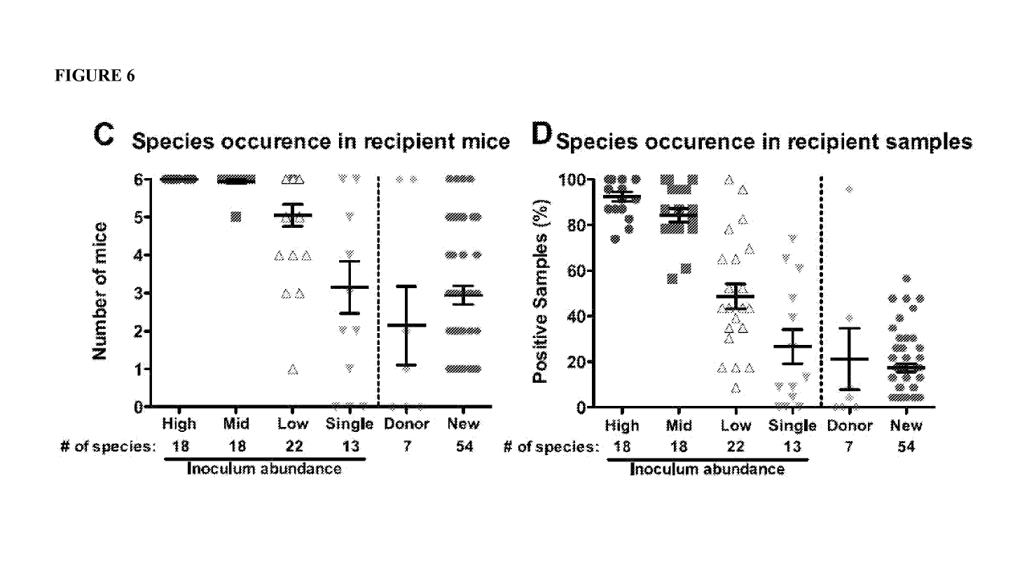

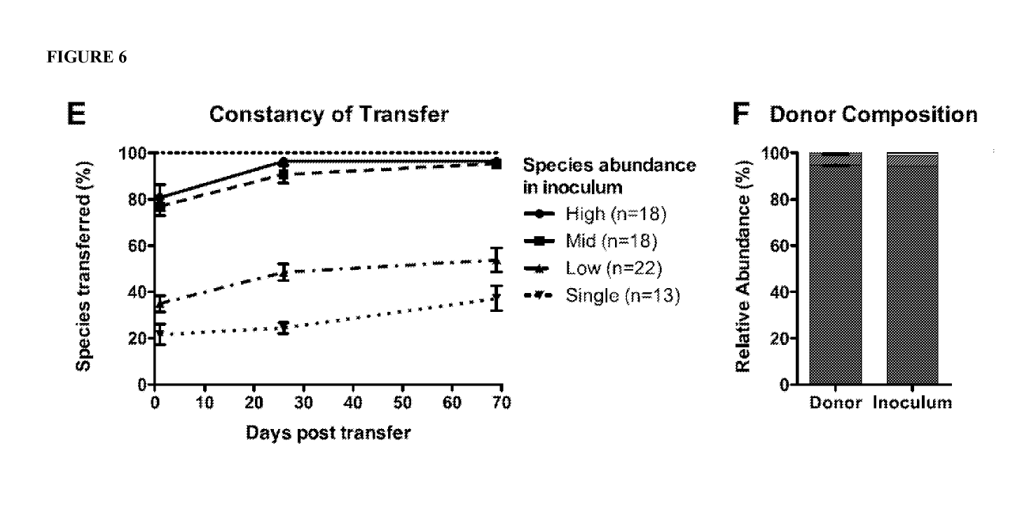

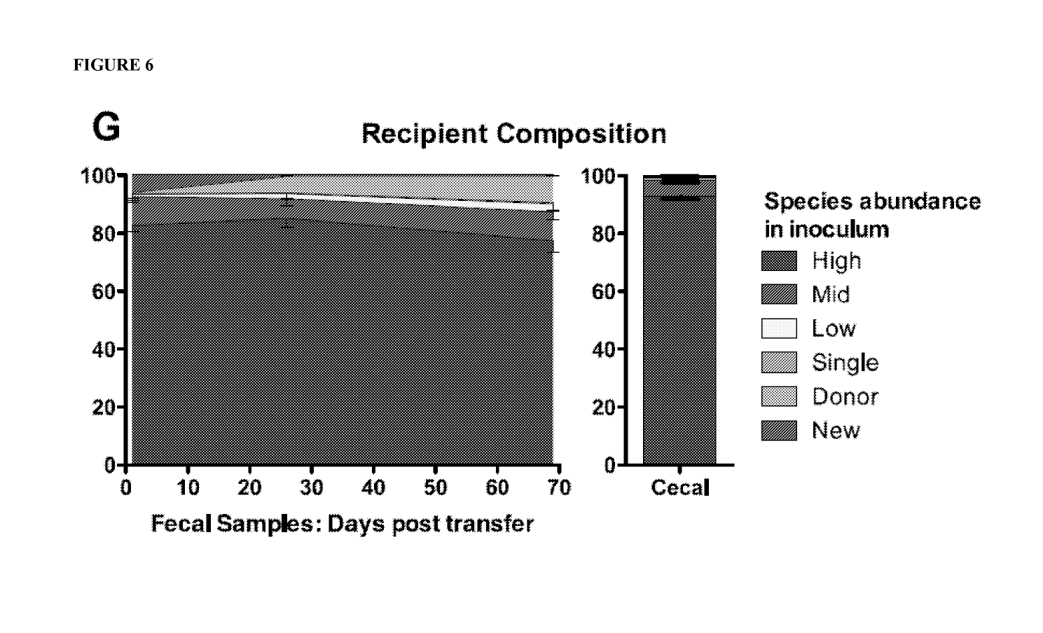

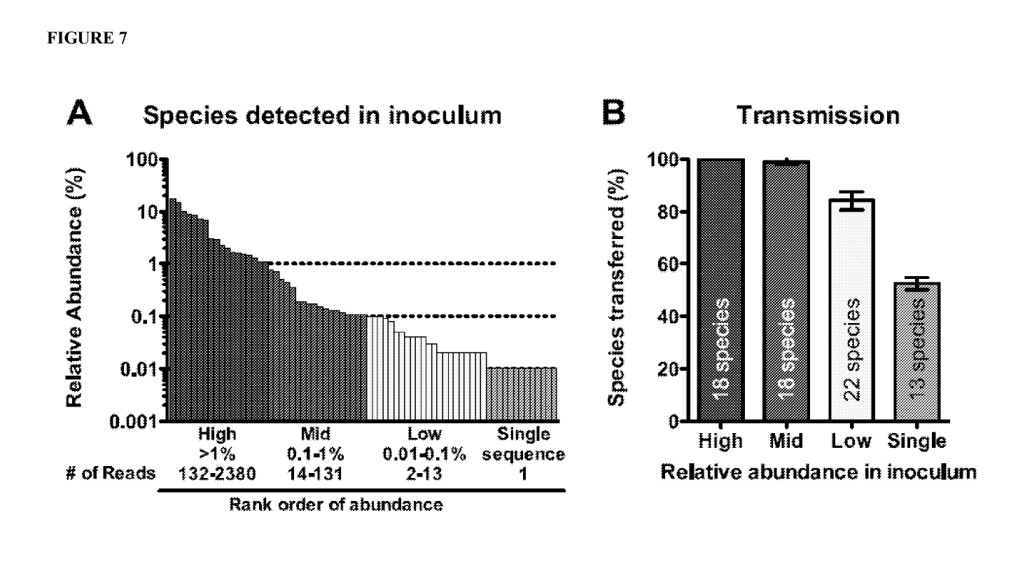

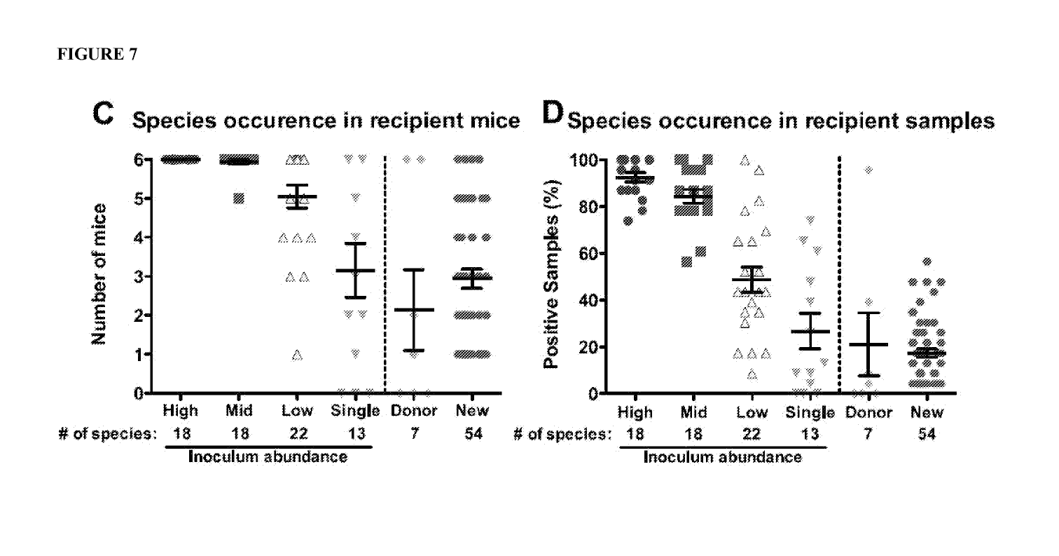

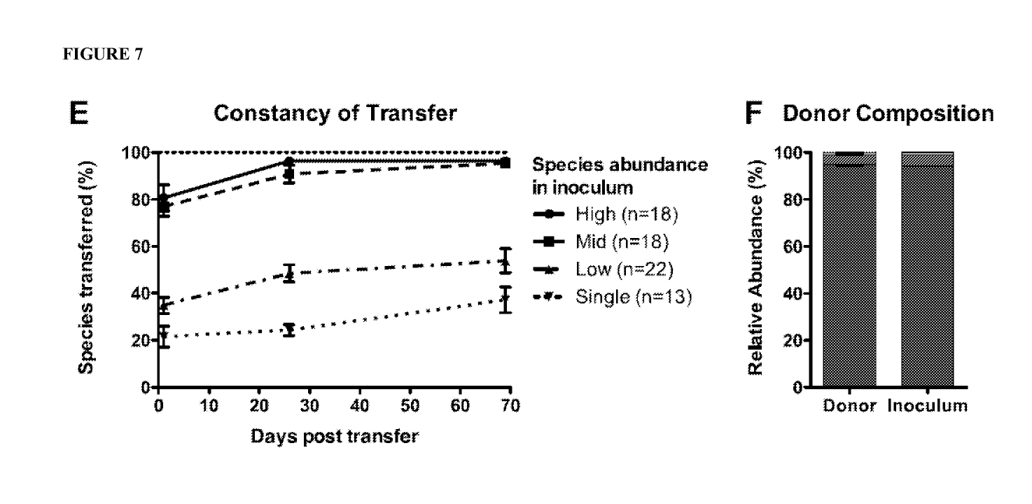

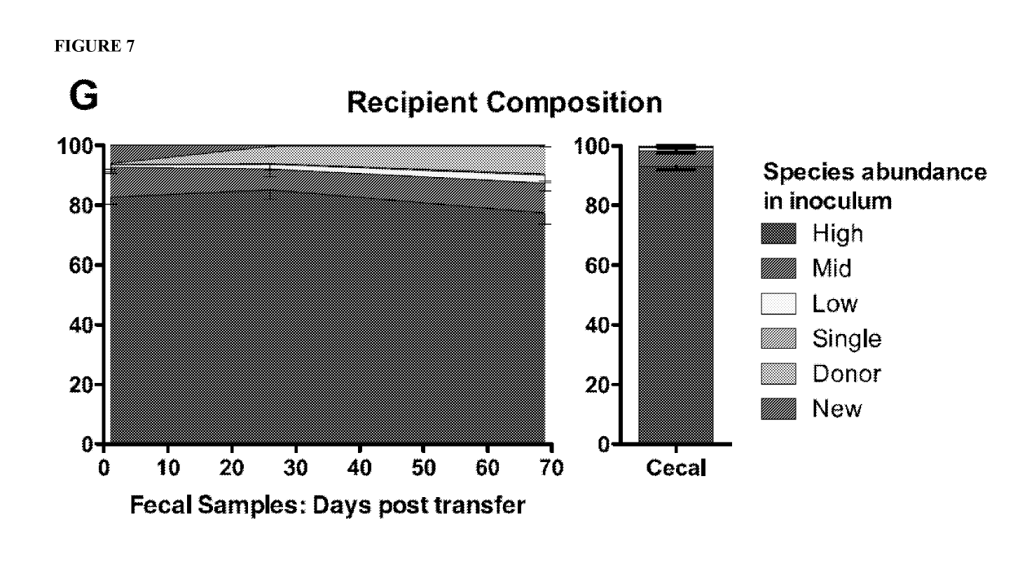

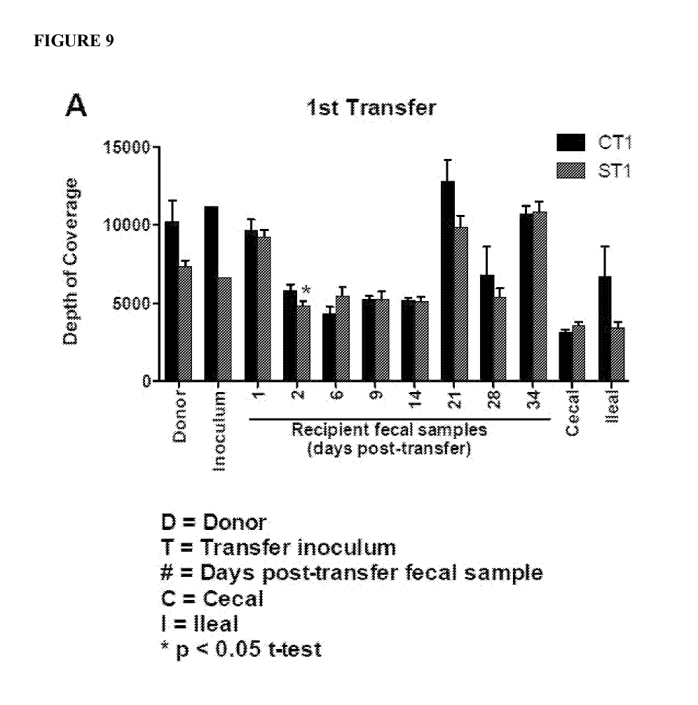

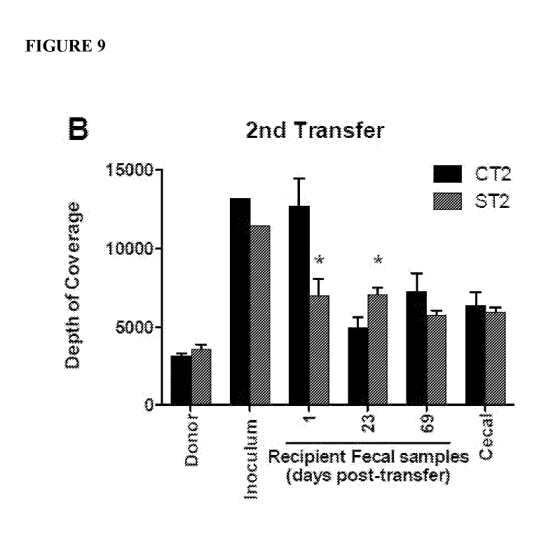

FIGS. 4A-G show microbiota transfer efficiency with a manipulated-microbiota donor source to germ-free mice. 3 conventional C57BL6 mice received STAT for 18 weeks, then cecal microbiota was collected in anaerobic transport media, mixed in reduced (no oxygen), sterile saline in an anaerobic chamber, and transferred to 8 germ-free Swiss-Webster mice. The donor cecal samples, the inoculum, and recipient intestinal microbiota was assessed by 16S rRNA high-throughput sequencing. Transfer of microbiota from mice receiving sub-therapeutic antibiotic treatment showed many of the same patterns as the control microbiota transfer (FIG. 3), including high transfer efficiency in the species that had high, mid, and low abundance in the inoculum, and greater than 50% transfer efficiency of organisms detected by only a single read. Follow transfer, organisms that were dominant in the donor and inoculum microbiota were dominant in the recipient microbiota. However, the major difference is that fewer species were detected in the inoculum (50 compared to 72), which may have been from reduced sequencing coverage (FIG. 9). Overall transfer efficiency patterns were also conserved upon a second transfer to a new set of germ free mice with microbiota from the first control recipients (FIG. 6) and from the transfer of microbiota from the first transfer (FIG. 7).

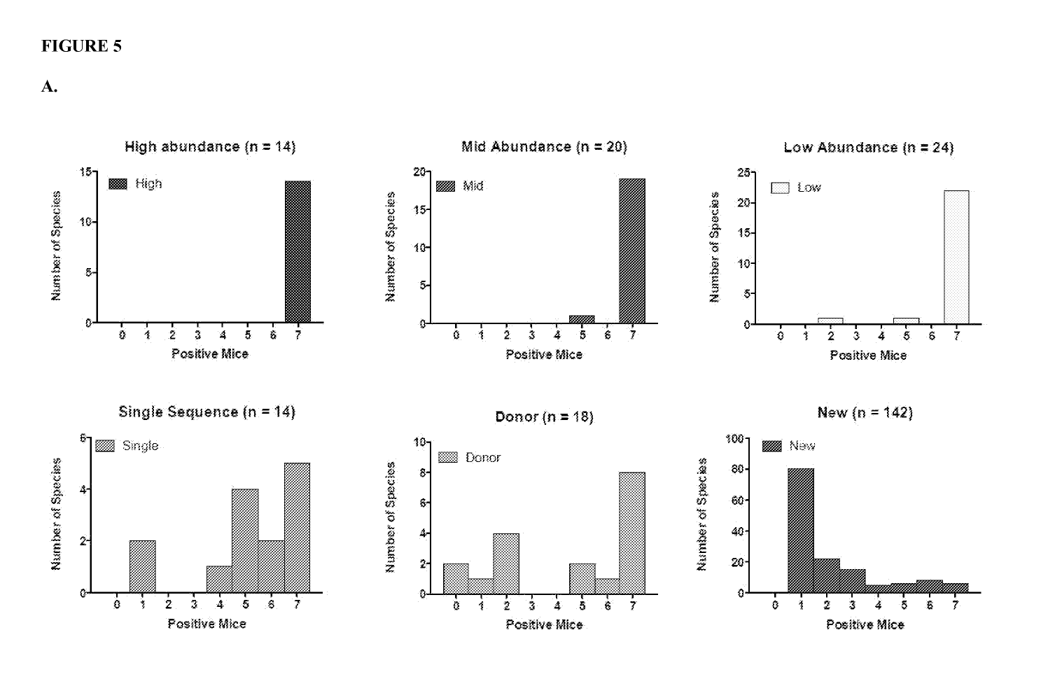

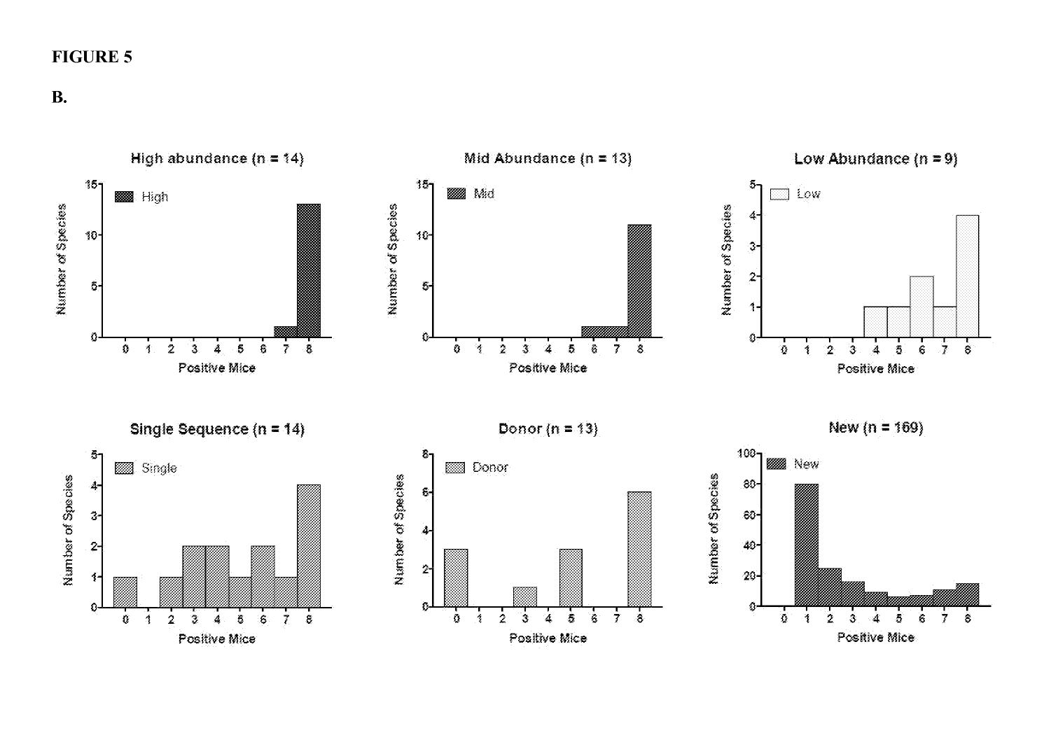

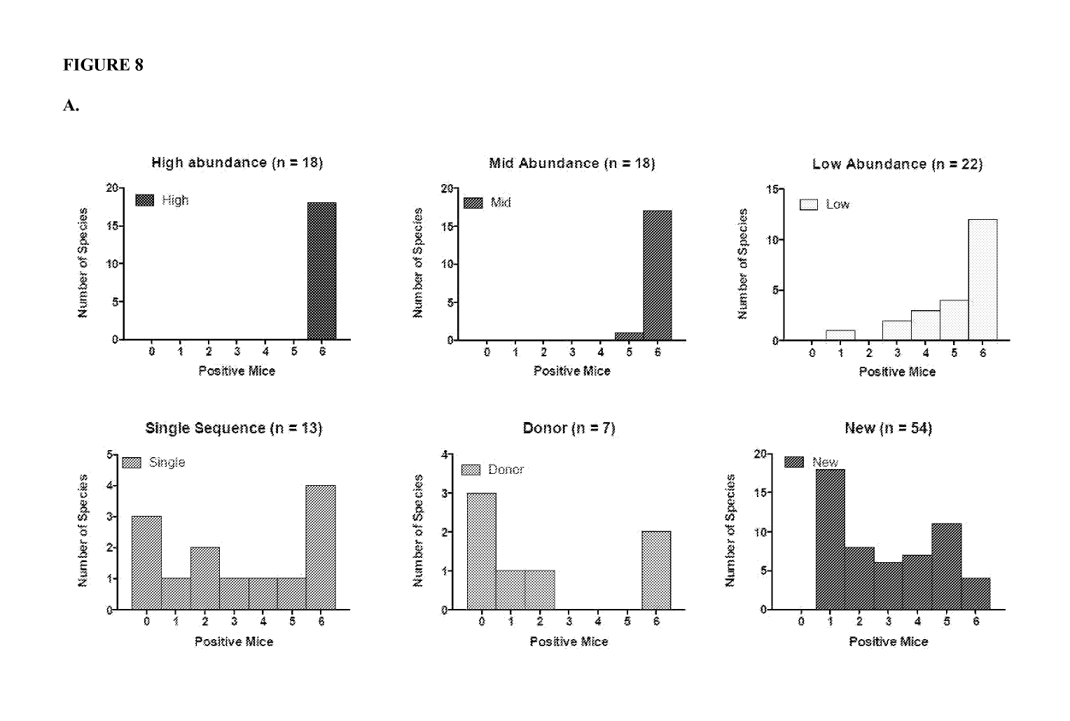

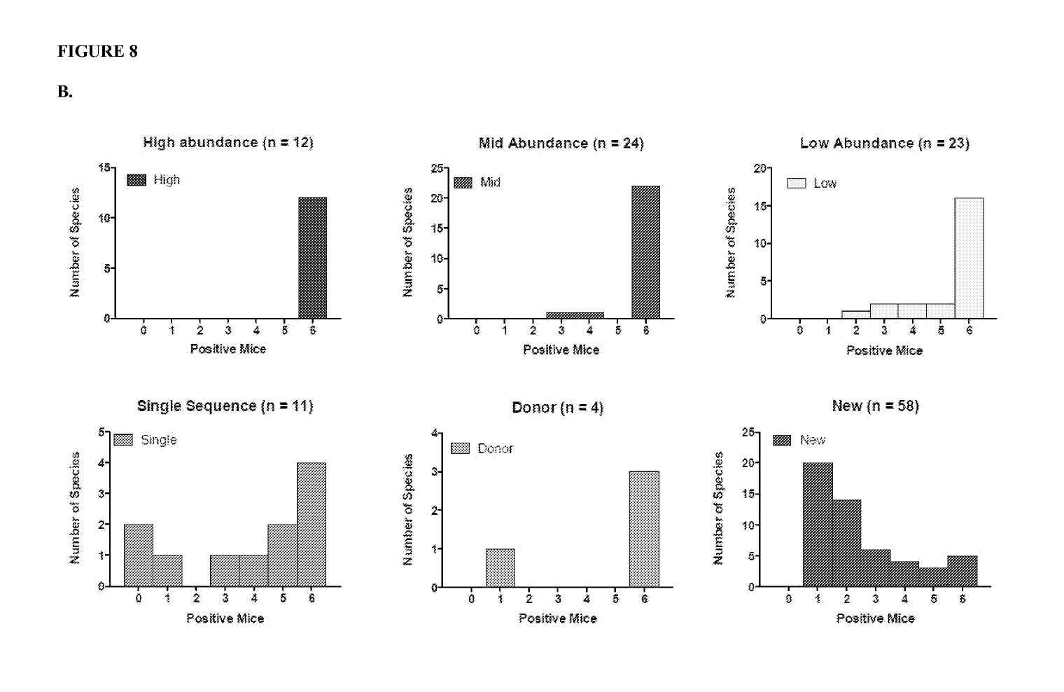

FIGS. 5A-B and 8A-B show distribution of inoculum species transmissibility. Histogram of species transmission into recipient mice for species with high abundance, mid abundance, low abundance, or detected by a single read in the inoculum, or detected in the individual donor specimens but not in the pooled inoculum, or new: not detected in the donor or inoculum samples. Detection of species in the recipient microbiota depends on the depth of sequencing (FIG. 8). Species with high, mid, and low abundance were detected in most recipient mice, and were effectively transferred. Species detected only by a single read, detected in the individual donor but not inoculum, or new species, display a bimodal distribution where some species appear in all recipients, and other species appear in only one recipient. If a species is present at 0.01% of the population, it theorhetically would be detected only by a single sequence in an inoculum sequenced at a depth of 1,000, however, random chance, PCR amplification bias, and sequencing bias can decrease the probability of detecting a species with low abundance. Conversely, there are some sequences that represent misidentification or contamination, which also would only be detected at low levels. Species detected only by a single read in the inoculum, detected in the individual donors, or new species that appear in a high proportion of the mice (5 or more) represent species that are likely actually present in the community, but at low abundance, while species in those same categories only detected in 1 to 2 mice are likely contaminants or sequencing noise. Thus, this data reveals that the species detected by only a single read in the inoculum, detected in the individual donor, or new species detected in the recipient, represent real species present and false findings from sequencing noise, thus the lower rates of transfer efficiency detected in the lowest categories are skewed by artifacts introduced by the sequencing technology.

FIGS. 9A-B show depth of coverage in microbiome transfer and recipient samples. (A) Number of 16S rRNA microbial sequences surveyed in the individual donor samples (n=3), the pooled inoculum (n=1), and the recipient fecal, cecal, and ileal samples in control (CT1, n=7), and STAT (ST1, n=8) germ-free microbiota-recipients. (A) Number of 16S rRNA microbial sequences surveyed in the individual donor samples (n=3, coming from CT1 or ST1 mice), the pooled inoculum (n=1), and the recipient fecal, cecal, and ileal samples in control (CT2, n=6), and STAT (ST2, n=6) germ-free microbiota-recipients.

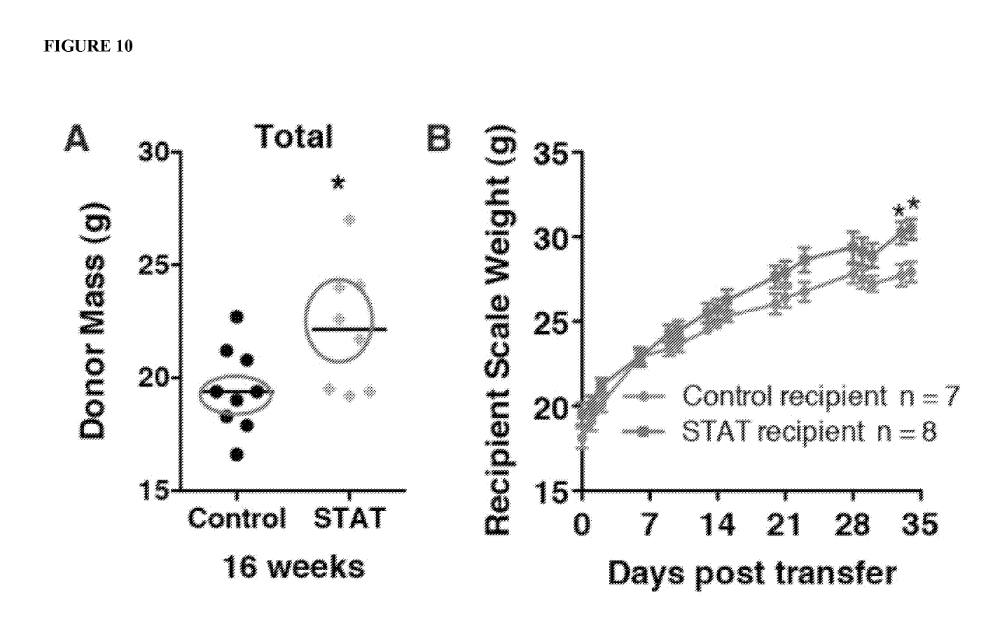

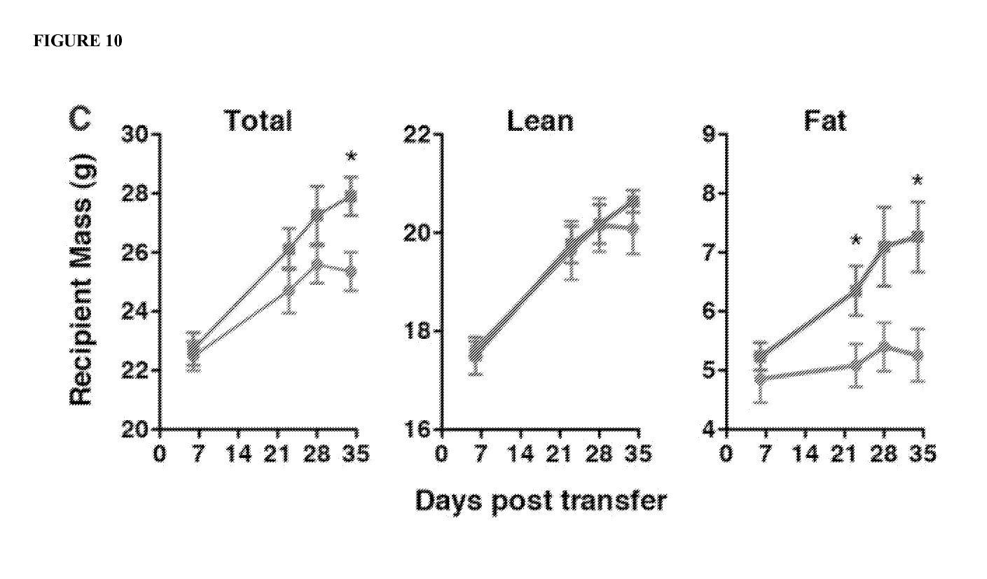

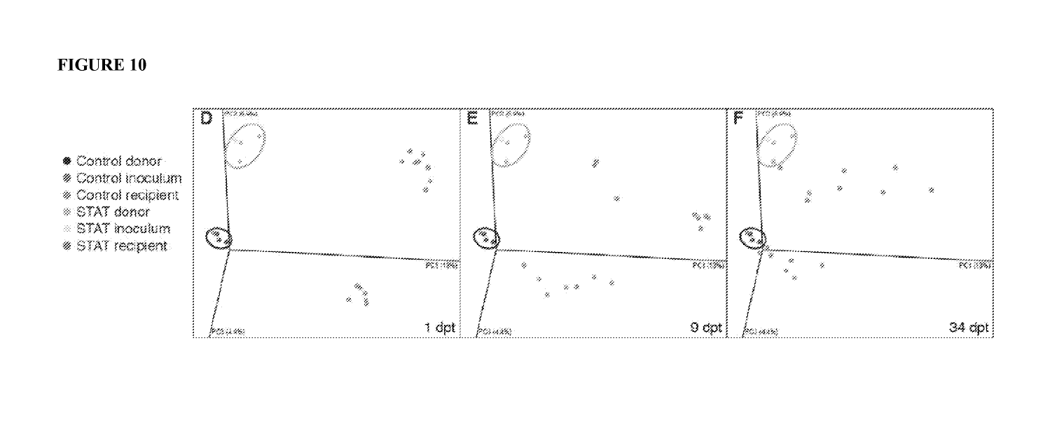

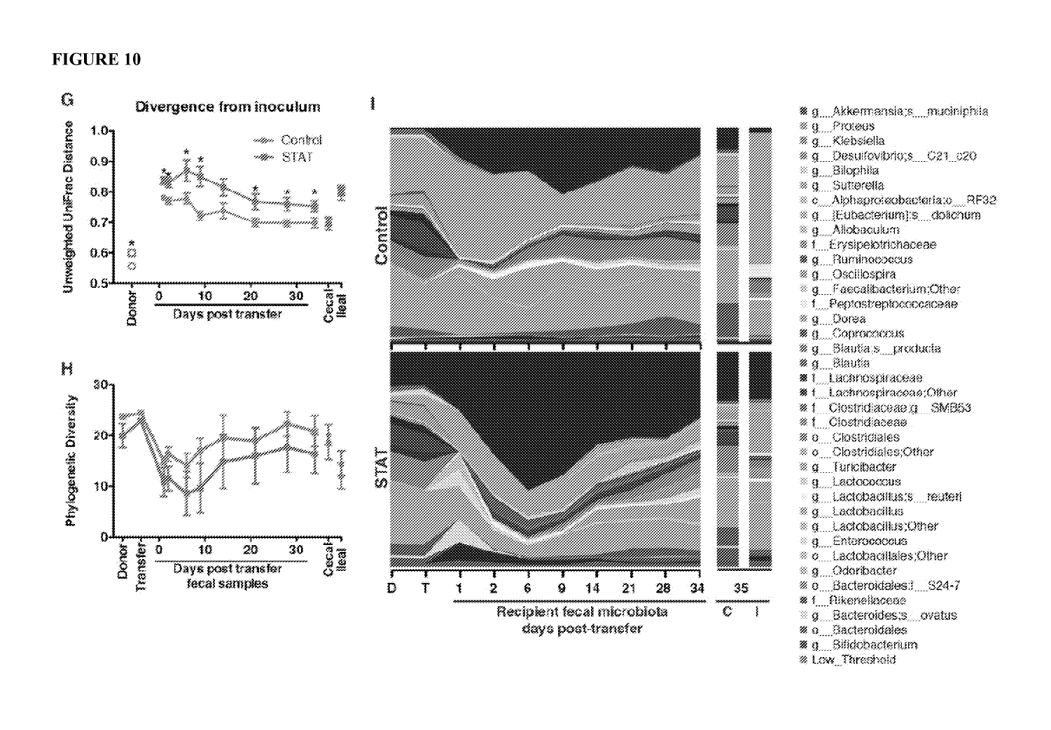

FIGS. 10A-I show metabolic and ecological consequences of transferring STAT microbiota. Cecal microbiota from 3 control and 3 STAT C57B/L6J mice at 18 weeks of age were collected, pooled in a saline solution, and transferred to 3-week old germ-free Swiss-Webster mice by oral gavage. Microbiota donors were selected based on the median total mass determined by DEXA scanning at 16-weeks (FIG. 10A). Scale weight of recipient mice was elevated in STAT-recipients over time (FIG. 10B). Total mass and fat mass in was elevated in conventionalized germ-free STAT-recipient mice (FIG. 10C), demonstrating that the obese associated microbiota is sufficient to transfer the obesity phenotype and the lean associated microbiota is sufficient to transfer the lean phenotype Community structure assessed by PCoA of unweighted UniFrac distances of the donor cecal, the transferred inoculum, and the recipient mouse fecal samples remained distinct over time between control and STAT recipients, demonstrating that the specific inoculum comprises a specific microbial community (FIG. 10D-F) 1 day following transfer, divergence from inoculum increased, but began to decrease after 9-days post transfer in both the STAT and control microbiota recipients. However, the control microbiota shows less divergence from inoculum than the STAT microbiota recipients, demonstrating that microbial community reassembly is more effective when the initial community is not under selective a disruptive selective pressure (FIG. 10G). Control recipient mice had higher phyogenetic diversity (FIG. 10H). Taxonomic representation differed between control and STAT recipients over time (FIG. 10I).

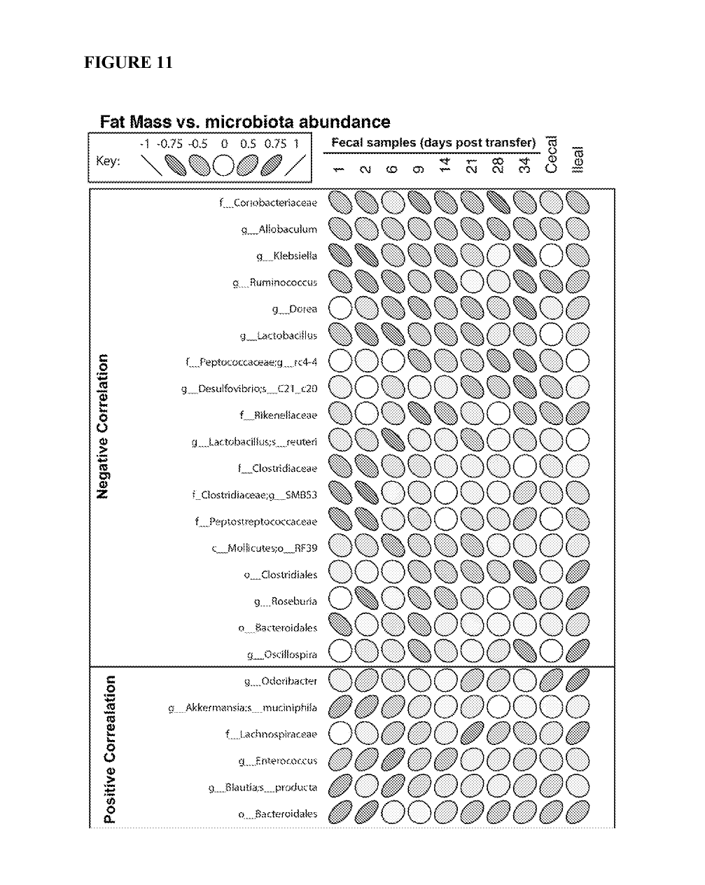

FIG. 11 shows microbial correlations with fat mass. Germ-free Swiss Webster mice were colonized with microbiota from Control or STAT mice. The intestinal microbiota of the recipients was surveyed over time (1-34-days post-transfer fecal specimens, cecal and ileal specimens 35-days post-transfer) by high throughput sequencing at an mean±SD depth of 6729±3334 sequences per sample. Taxonomic assignment used the QIIME pipeline based on the May 20, 2013 Green Genes database of 16S microbial sequences. The Spearman correlation was calculated with reference to fat mass at 34-days-post transfer with relative abundance of the predominant species (>1% in any sample). Microbiota with at least one significant correlation (p<0.05), and consistent correlation direction are shown. An ellipse with a forward slant represents a positive Spearman correlation, and a backwards slant represents a negative Spearman correlation, and the narrowness of the ellipse indicates the strength of the correlation (higher rho value). Microbiota are reported at the lowest possible identifiable level, indicated by the letter preceding the underscore: o=order, f=family, g=genus, s=species. This example defines the significant taxa to the genus level in most cases, and including the species level, and represents candidate microbiota for manipulating fat mass, extending the observations in Table 6.

In the absence of any further perturbation, this work characterizes which bacteria can successfully colonize new hosts and dominate the new environmental niche that the uncolonized gut represents. The results show that although there is an initial change in the balance of dominant organisms, there is extensive transfer that populates the formerly germ-free niche with a microbiota with similar composition to the donor microbiota.

TABLE 1
Summary of transfer efficiency and composition

| | | | | |
|---|---|---|---|---|
| | Number of | Transfer | | Recipient |
| | species in | efficiency (%) | Inoculum | proportion (%) |
| Representation in Inoculum | inoculum | (FIG. 3A) | proportion (%) | (FIG. 3C)^{b} |

| | | | | |
|---|---|---|---|---|
| High abundance (>1%) | 14 | 100.0 | 91.6 | 67.5 |
| Moderate abundance (0.1-1%) | 20 | 98.6 | 7.3 | 26.4 |
| Low abundance (0.01-0.1%) | 24 | 95.8 | 1.0 | 4.6 |
| Single read | 14 | 74.5 | 0.1 | 0.3 |
| Not in inoculum (new)^{a} | 0 | NA | 0 | 1.2 |

^{a}Newly detected; may represent true new, or below detection limit in donor
^{b}Across all time points

TABLE 2
Transfer efficiency in 7 germ-free microbiota recipients

| | Number of | | | | | | | | |
|---|---|---|---|---|---|---|---|---|---|
| Representation | species in | Percent of species detected in recipient mice | | | | | | | |
| in Inoculum | inoculum | CT1 | CT2 | CT3 | CT4 | CT5 | CT6 | CT7 | Mean |
| Highly abundant | 14 | 100.0 | 100.0 | 100.0 | 100.0 | 100.0 | 100.0 | 100.0 | 100.0 ± 0.0 |
| (>1%) | | | | | | | | | |
| Moderately | 20 | 95.0 | 100.0 | 95.0 | 100.0 | 100.0 | 100.0 | 100.0 | 98.6 ± 2.4 |
| abundant (0.1-1%) | | | | | | | | | |
| Low abundance | 24 | 95.8 | 100.0 | 95.8 | 95.8 | 100.0 | 91.7 | 91.7 | 95.8 ± 3.4 |
| (0.01-0.1%) | | | | | | | | | |
| Extremely low | 14 | 78.6 | 78.6 | 57.1 | 64.3 | 85.7 | 64.3 | 92.9 | 74.5 ± 12.9 |
| abundance (<0.01%) | | | | | | | | | |

TABLE 3
Transfer efficiency in 7 germ-free microbiota recipients

| | Number of | | | | | | | | |
|---|---|---|---|---|---|---|---|---|---|
| Representation | species in | Number of species detected in recipient mice | | | | | | | |
| in Inoculum | inoculum | CT1 | CT2 | CT3 | CT4 | CT5 | CT6 | CT7 | Mean |
| Highly abundant | 14 | 14 | 14 | 14 | 14 | 14 | 14 | 14 | 14.0 ± 0.0 |
| (>1%) | | | | | | | | | |
| Moderately | 20 | 19 | 20 | 19 | 20 | 20 | 20 | 20 | 19.7 ± 0.5 |
| abundant (0.1-1%) | | | | | | | | | |
| Low abundance | 24 | 23 | 24 | 23 | 23 | 24 | 22 | 22 | 23.0 ± 0.8 |
| (0.01-0.1%) | | | | | | | | | |
| Extremely low | 14 | 11 | 11 | 8 | 9 | 12 | 9 | 13 | 10.4 ± 1.8 |
| abundance | | | | | | | | | |
| (<0.01%) | | | | | | | | | |
| Not in inoculum | 0 | 49 | 47 | 51 | 38 | 51 | 74 | 80 | 55.7 ± 15.3 |
| (new) | | | | | | | | | |

TABLE 4
Transfer composition in 7 germ-free microbiota recipients

| | Inoculum | | | | | | | | |
|---|---|---|---|---|---|---|---|---|---|
| Representation | proportion | Percent of recipient microbiota | | | | | | | |
| in Inoculum | (%) | CT1 | CT2 | CT3 | CT4 | CT5 | CT6 | CT7 | Mean |
| Highly abundant | 91.56 | 73.3 | 76.0 | 54.5 | 73.2 | 69.9 | 64.7 | 61.0 | 67.5 ± 7.8 |
| (>1%) | | | | | | | | | |
| Moderately | 7.34 | 20.7 | 19.1 | 37.2 | 21.6 | 24.6 | 29.7 | 31.9 | 26.4 ± 6.7 |
| abundant (0.1-1%) | | | | | | | | | |
| Low abundance | 0.98 | 5.3 | 3.9 | 5.1 | 4.7 | 4.1 | 3.8 | 5.6 | 4.6 ± 0.7 |
| (0.01-0.1%) | | | | | | | | | |
| Extremely low | 0.13 | 0.3 | 0.5 | 0.2 | 0.1 | 0.3 | 0.2 | 0.3 | 0.3 ± 0.1 |
| abundance | | | | | | | | | |
| (<0.01%) | | | | | | | | | |
| Not in inoculum | 0.00 | 0.4 | 0.5 | 3.0 | 0.4 | 1.1 | 1.6 | 1.3 | 1.2 ± 0.9 |
| (new) | | | | | | | | | |

TABLE 5
Transfer efficiency of all species detected in the control inoculum. Relative abundance in the 3 individual donor
samples, the 1 inoculum that was transferred, and the 69 recipient fecal samples taken from 7 mice over time, number
of mice in which the inoculum species was detected in and % of the recipient samples it was detected in.

| | | | | # | % |
|---|---|---|---|---|---|
| Bacteria | Donor | Inoculum | Recipient | Mice | Samples |
| p_Firmicutes; c_Erysipelotrichi; o_Erysipelotrichales; | 26.06% | 24.30% | 25.73% | 7 | 100.0% |
| f_Erysipelotrichaceae; g_Allobaculum; s_{—} | | | | | |
| p_Bacteroidetes; c_Bacteroidia; o_Bacteroidales; | 26.11% | 19.90% | 17.08% | 7 | 100.0% |
| f_S24-7; g_; s_{—} | | | | | |
| p_Firmicutes; c_Clostridia; o_Clostridiales; f_{—} | 9.95% | 12.59% | 1.67% | 7 | 97.1% |
| Lachnospiraceae; g_; s_{—} | | | | | |
| p_Firmicutes; c_Clostridia; o_Clostridiales; f_; g_; s_{—} | 5.82% | 7.58% | 4.39% | 7 | 94.2% |
| p_Firmicutes; c_Clostridia; o_Clostridiales; f_{—} | 3.59% | 5.72% | 1.01% | 7 | 78.3% |
| Ruminococcaceae; g_Oscillospira; s_{—} | | | | | |
| p_Firmicutes; c_Clostridia; o_Clostridiales; f_{—} | 2.29% | 4.15% | 0.33% | 7 | 87.0% |
| Lachnospiraceae; g_Coprococcus; s_{—} | | | | | |
| p_Firmicutes; c_Clostridia; o_Clostridiales; f_{—} | 4.15% | 3.11% | 2.40% | 7 | 95.7% |
| Clostridiaceae; g_; s_{—} | | | | | |
| p_Firmicutes; c_Clostridia; o_Clostridiales; f_{—} | 1.78% | 2.99% | 0.46% | 7 | 88.4% |
| Lachnospiraceae; Other; Other | | | | | |
| p_Bacteroidetes; c_Bacteroidia; o_Bacteroidales; | 5.16% | 2.91% | 4.43% | 7 | 98.6% |
| f_Rikenellaceae; g_; s_{—} | | | | | |
| p_Firmicutes; c_Clostridia; o_Clostridiales; f_{—} | 1.66% | 2.10% | 1.72% | 7 | 95.7% |
| Lachnospiraceae; g_Dorea; s | | | | | |
| p_Proteobacteria; c_Deltaproteobacteria; o_{—} | 1.73% | 2.05% | 0.39% | 7 | 78.3% |
| Desulfovibrionales; f_Desulfovibrionaceae; g_{—} | | | | | |
| Desulfovibrio; s_C21_c20 | | | | | |
| p_Firmicutes; c_Clostridia; o_Clostridiales; f_{—} | 0.66% | 1.46% | 0.16% | 7 | 85.5% |
| Ruminococcaceae; g_Ruminococcus; s_{—} | | | | | |
| p_Firmicutes; c_Bacilli; o_Lactobacillales; f_{—} | 1.32% | 1.45% | 7.48% | 7 | 98.6% |
| Lactobacillaceae; g_Lactobacillus; s_{—} | | | | | |
| p_Proteobacteria; c_Deltaproteobacteria; o_{—} | 1.44% | 1.24% | 0.20% | 7 | 68.1% |
| Desulfovibrionales; f_Desulfovibrionaceae; g_{—} | | | | | |
| Bilophila; s_{—} | | | | | |
| p_Firmicutes; c_Bacilli; o_Turicibacterales; f_{—} | 0.73% | 0.96% | 0.58% | 7 | 72.5% |
| Turicibacteraceae; g_Turicibacter; s_{—} | | | | | |
| p_Tenericutes; c_Mollicutes; o_Anaeroplasmatales; | 0.42% | 0.72% | 0.01% | 5 | 14.5% |
| f_Anaeroplasmataceae; g_Anaeroplasma; s_{—} | | | | | |
| p_Firmicutes; c_Clostridia; o_Clostridiales; f_{—} | 0.38% | 0.62% | 1.11% | 7 | 95.7% |
| Peptostreptococcaceae; g_; s_{—} | | | | | |
| p_Actinobacteria; c_Actinobacteria; o_Bifidobacteriales; | 0.16% | 0.60% | 0.99% | 7 | 95.7% |
| f_Bifidobacteriaceae; g_Bifidobacterium; s_{—} | | | | | |
| p_Firmicutes; c_Clostridia; o_Clostridiales; Other; Other; Other | 0.50% | 0.56% | 0.72% | 7 | 100.0% |
| p_Firmicutes; c_Clostridia; o_Clostridiales; f_{—} | 0.36% | 0.52% | 0.13% | 7 | 78.3% |
| Lachnospiraceae; g_[Ruminococcus]; s_gnavus | | | | | |
| p_Firmicutes; c_Clostridia; o_Clostridiales; f_{—} | 0.43% | 0.51% | 0.21% | 7 | 79.7% |
| Ruminococcaceae; g_; s_{—} | | | | | |
| p_Bacteroidetes; c_Bacteroidia; | 0.42% | 0.48% | 0.01% | 7 | 36.2% |
| o_Bacteroidales; f_Prevotellaceae; g_Prevotella; s_{—} | | | | | |
| p_Verrucomicrobia; c_Verrucomicrobiae; o_{—} | 0.93% | 0.45% | 17.44% | 7 | 98.6% |
| Verrucomicrobiales; f_Verrucomicrobiaceae; g_{—} | | | | | |
| Akkermansia; s_muciniphila | | | | | |
| p_Bacteroidetes; c_Bacteroidia; o_Bacteroidales; | 0.91% | 0.34% | 1.05% | 7 | 84.1% |
| f_[Odoribacteraceae]; g_Odoribacter; s_{—} | | | | | |
| p_Bacteroidetes; c_Bacteroidia; o_Bacteroidales; | 0.51% | 0.28% | 0.27% | 7 | 88.4% |
| f_Bacteroidaceae; g_Bacteroides; s_ovatus | | | | | |
| p_Firmicutes; c_Erysipelotrichi; o_Erysipelotrichales; | 0.16% | 0.20% | 1.91% | 7 | 97.1% |
| f_Erysipelotrichaceae; g_; s_{—} | | | | | |
| p_Firmicutes; c_Clostridia; o_Clostridiales; f_{—} | 0.16% | 0.17% | 0.31% | 7 | 91.3% |
| Clostridiaceae; g_SMB53; s_{—} | | | | | |
| p_Firmicutes; c_Clostridia; o_Clostridiales; f_{—} | 0.12% | 0.17% | 0.11% | 7 | 84.1% |
| Clostridiaceae; Other; Other | | | | | |
| p_Firmicutes; Other; Other; Other; Other; Other | 0.12% | 0.16% | 0.13% | 7 | 91.3% |
| p_Firmicutes; c_Clostridia; o_Clostridiales; f_{—} | 0.12% | 0.15% | 0.13% | 7 | 75.4% |
| [Mogibacteriaceae]; g_; s_{—} | | | | | |
| p_Firmicutes; c_Clostridia; o_Clostridiales; f_{—} | 0.12% | 0.13% | 0.04% | 7 | 71.0% |
| Ruminococcaceae; Other; Other | | | | | |
| p_Firmicutes; c_Clostridia; o_Clostridiales; f_{—} | 0.06% | 0.12% | 0.03% | 7 | 36.2% |
| Ruminococcaceae; g_Anaerofilum; s_{—} | | | | | |
| p_Firmicutes; c_Bacilli; o_Lactobacillales; f_{—} | 0.20% | 0.11% | 1.28% | 7 | 94.2% |
| Lactobacillaceae; g_Lactobacillus; s_reuteri | | | | | |
| p_Actinobacteria; c_Coriobacteriia; o_Coriobacteriales; | 0.02% | 0.11% | 0.03% | 7 | 69.6% |
| f_Coriobacteriaceae; g_Adlercreutzia; s_{—} | | | | | |
| p_Bacteroidetes; c_Bacteroidia; o_Bacteroidales; | 0.10% | 0.10% | 0.07% | 7 | 84.1% |
| Other; Other; Other | | | | | |
| Bacteria: Other | 0.10% | 0.10% | 0.06% | 7 | 85.5% |
| p_Firmicutes; c_Clostridia; o_Clostridiales; f_{—} | 0.08% | 0.09% | 0.02% | 7 | 50.7% |
| Dehalobacteriaceae; g_Dehalobacterium; s_{—} | | | | | |
| p_Proteobacteria; c_Betaproteobacteria; o_{—} | 0.17% | 0.08% | 1.46% | 7 | 94.2% |
| Burkholderiales; f_Alcaligenaceae; g_Sutterella; s_{—} | | | | | |
| p_Firmicutes; c_Clostridia; o_Clostridiales; f_{—} | 0.01% | 0.06% | 0.03% | 7 | 43.5% |
| Peptococcaceae; g_; s_{—} | | | | | |
| p_Cyanobacteria; c_ 4C0d-2; o_YS2; f_; g_; s_{—} | 0.03% | 0.05% | 0.08% | 7 | 56.5% |
| p_Tenericutes; c_Mollicutes; o_RF39; f_; g_; s_{—} | 0.15% | 0.04% | 0.21% | 7 | 73.9% |
| p_Firmicutes; c_Clostridia; o_Clostridiales; f_{—} | 0.12% | 0.04% | 0.03% | 7 | 40.6% |
| Lachnospiraceae; g_Anaerostipes; s_{—} | | | | | |
| p_Bacteroidetes; Other; Other; Other; Other; Other | 0.04% | 0.04% | 0.01% | 7 | 52.2% |
| p_Proteobacteria; c_Alphaproteobacteria; o_{—} | 0.01% | 0.04% | 0.04% | 7 | 52.2% |
| RF32; f_; g_; s_{—} | | | | | |
| p_Actinobacteria; c_Coriobacteriia; | 0.02% | 0.04% | 0.28% | 7 | 78.3% |
| o_Coriob acteriales; f_Coriobacteriaceae; g_; s_{—} | | | | | |
| p_Firmicutes; c_Bacilli; o_Lactobacillales; f_{—} | 0.05% | 0.03% | 0.35% | 7 | 87.0% |
| Lactobacillaceae; g_Lactobacillus; Other | | | | | |
| p_Firmicutes; c_Erysipelotrichi; o_Erysipelotrichales; | 0.04% | 0.03% | 0.17% | 7 | 92.8% |
| f_Erysipelotrichaceae; Other; Other | | | | | |
| p_Proteobacteria; c_Deltaproteobacteria; o_{—} | 0.03% | 0.03% | 0.00% | 5 | 10.1% |
| Desulfovibrionales; f_Desulfovibrionaceae; Other; Other | | | | | |
| p_Proteobacteria; Other; Other; Other; Other; Other | 0.02% | 0.03% | 0.01% | 7 | 23.2% |
| p_Firmicutes; c_Bacilli; o_Lactobacillales; f_{—} | 0.02% | 0.03% | 1.03% | 7 | 94.2% |
| Streptococcaceae; g_Lactococcus; s_{—} | | | | | |
| p_Bacteroidetes; c_Bacteroidia; o_Bacteroidales; | 0.07% | 0.02% | 0.04% | 7 | 62.3% |
| f_Bacteroidaceae; g_Bacteroides; s_acidifaciens | | | | | |
| p_Firmicutes; c_Bacilli; o_Lactobacillales; f_{—} | 0.03% | 0.02% | 0.16% | 7 | 84.1% |
| Lactobacillaceae; g_Lactobacillus; s_vaginalis | | | | | |
| p_Firmicutes; c_Clostridia; o_Clostridiales; f_{—} | 0.02% | 0.02% | 0.07% | 7 | 31.9% |
| Peptococcaceae; g_rc4-4; s_{—} | | | | | |
| p_Firmicutes; c_Clostridia; Other; Other; Other; Other | 0.02% | 0.02% | 0.01% | 7 | 36.2% |
| p_Actinobacteria; c_Actinobacteria; o_Bifidobacteriales; | 0.01% | 0.02% | 0.03% | 7 | 58.0% |
| f_Bifidobacteriaceae; g_Bifidobacterium; Other | | | | | |
| p_Firmicutes; c_Clostridia; o_Clostridiales; f_{—} | 0.01% | 0.02% | 0.00% | 2 | 5.8% |
| Clostridiaceae; g_02d06; s_{—} | | | | | |
| p_Firmicutes; c_Clostridia; o_Clostridiales; f_{—} | 0.01% | 0.02% | 0.40% | 7 | 71.0% |
| Ruminococcaceae; g_Faecalibacterium; Other | | | | | |
| p_Actinobacteria; c_Actinobacteria; o_Bifidobacteriales; | 0.01% | 0.02% | 0.04% | 7 | 69.6% |
| f_Bifidobacteriaceae; g_Bifidobacterium; s_pseudolongum | | | | | |
| p_Firmicutes; c_Clostridia; o_Clostridiales; f_{—} | 0.06% | 0.01% | 0.15% | 7 | 65.2% |
| Lachnospiraceae; g_Roseburia; s_{—} | | | | | |
| p_Firmicutes; c_Clostridia; o_Clostridiales; f_{—} | 0.03% | 0.01% | 0.00% | 5 | 18.8% |
| Lachnospiraceae; g_Coprococcus; Other | | | | | |
| p_Bacteroidetes; c_Bacteroidia; o_Bacteroidales; | 0.02% | 0.01% | 0.00% | 1 | 1.4% |
| f_Prevotellaceae; g_Prevotella; Other | | | | | |
| p_Firmicutes; c_Clostridia; o_Clostridiales; f_{—} | 0.02% | 0.01% | 0.01% | 7 | 26.1% |
| Christensenellaceae; g_; s_{—} | | | | | |
| p_Firmicutes; c_Clostridia; o_Clostridiales; f_{—} | 0.01% | 0.01% | 0.00% | 5 | 13.0% |
| [Mogibacteriaceae]; Other; Other | | | | | |
| p_Firmicutes; c_Bacilli; o_Lactobacillales; f_{—} | 0.01% | 0.01% | 0.02% | 7 | 65.2% |
| Lactobacillaceae; g_Lactobacillus; s_delbrueckii | | | | | |
| p_Firmicutes; c_Clostridia; o_Clostridiales; f_{—} | 0.01% | 0.01% | 0.01% | 6 | 36.2% |
| Clostridiaceae; g_Clostridium; Other | | | | | |
| p_Proteobacteria; c_Deltaproteobacteria; o_{—} | 0.01% | 0.01% | 0.00% | 1 | 1.4% |
| Desulfovibrionales; Other; Other; Other | | | | | |
| p_Firmicutes; c_Clostridia; o_Clostridiales; f_{—} | 0.01% | 0.01% | 0.00% | 6 | 15.9% |
| Lachnospiraceae; g_Roseburia; Other | | | | | |
| p_Bacteroidetes; c_Bacteroidia; o_Bacteroidales; | 0.01% | 0.01% | 0.01% | 7 | 21.7% |
| f_Bacteroidaceae; g_Bacteroides; s_{—} | | | | | |
| p_Firmicutes; c_Clostridia; o_Clostridiales; f_{—} | 0.01% | 0.01% | 0.01% | 5 | 24.6% |
| Peptostreptococcaceae; Other; Other | | | | | |
| p_Firmicutes; c_Clostridia; o_Clostridiales; f_{—} | 0.01% | 0.01% | 0.00% | 5 | 14.5% |
| Ruminococcaceae; g_Oscillospira; Other | | | | | |
| p_Firmicutes; c_Bacilli; o_Lactobacillales; f_{—} | 0.00% | 0.01% | 0.04% | 7 | 60.9% |
| Enterococcaceae; g_Enterococcus; s_{—} | | | | | |
| p_Firmicutes; c_Clostridia; o_Clostridiales; f_{—} | 0.00% | 0.01% | 0.00% | 4 | 10.1% |
| Ruminococcaceae; g_Oscillospira; s_guilliermondii | | | | | |

TABLE 6
Comparison between microbiota in control and STAT inoculum and donor samples. Since the transferred microbiota
induced a metabolic phenotype, the microorganisms present or overrepresented in the control inoculum and microbiota
donors are candidate microbiota to protect against obesity and the microorganisms present or overrepresented in
the STAT inoculum and microbiota donors are candidate microbiota that contribute to obesity, or possibly to weight
gain for malnourished individuals, or growth promotion for short stature children. The Table shows all bacteria identified
at their lowest possible taxonomic level in control and STAT inoculum. Columns 2 and 3 show the relative abundance (%)
of each taxa within the sample for the control inoculum and the STAT inoculum, respectively. Column 3 shows the fold-change
(STAT abundance/Control abundance) in which Absent means that the abundance in Control Inoculum is greater than in STAT
Inoculum. Column 4 converts fold-change to log₂-fold change.

| | Control | | | Log2 Fold |
|---|---|---|---|---|
| Taxon | Inoculum | STAT Inoculum | Fold- Change | Change |
| p_Actinobacteria; c_Actinobacteria; o_Bifidobacteriales; | 0.010%⁺ | 0.000% | Absent | Absent |
| f_Bifidobacteriaceae; g_Bifidobacterium; Other | | | | |
| p_Actinobacteria; c_Actinobacteria; o_Bifidobacteriales; | 0.006%⁺ | 0.000% | Absent | Absent |
| f_Bifidobacteriaceae; g_Bifidobacterium; s_adolescentis | | | | |
| p_Actinobacteria; c_Actinobacteria; o_Bifidobacteriales; | 0.005%⁺ | 0.000% | Absent | Absent |
| f_Bifidobacteriaceae; g_Bifidobacterium; s_pseudolongum | | | | |
| p_Actinobacteria; c_Coriobacteriia; o_Coriobacteriales; | 0.023%⁺ | 0.000% | Absent | Absent |
| f_Coriobacteriaceae; g_; s_{—} | | | | |
| p_Bacteroidetes; c_Bacteroidia; o_Bacteroidales; | 0.020%⁺ | 0.000% | Absent | Absent |
| f_Prevotellaceae; g_Prevotella; Other | | | | |
| p_Cyanobacteria; c_4C0d- 2; o_YS2; f_; g_; s_{—} | 0.031%⁺ | 0.000% | Absent | Absent |
| p_Firmicutes; c_Bacilli; o_Lactobacillales; f_Lactobacillaceae; | 0.014%⁺ | 0.000% | Absent | Absent |
| g_Lactobacillus; s_delbrueckii | | | | |
| p_Firmicutes; c_Bacilli; o_Lactobacillales; f_Lactobacillaceae; | 0.197%** | 0.000% | Absent | Absent |
| g_Lactobacillus; s_reuteri | | | | |
| p_Firmicutes; c_Bacilli; o_Lactobacillales; f_Lactobacillaceae; | 0.031%⁺ | 0.000% | Absent | Absent |
| g_Lactobacillus; s_vaginalis | | | | |
| p_Firmicutes; c_Bacilli; o_Lactobacillales; | 0.003%⁺ | 0.000% | Absent | Absent |
| f_Streptococcaceae; Other; Other | | | | |
| p_Firmicutes; c_Bacilli; o_Lactobacillales; | 0.020%⁺ | 0.000% | Absent | Absent |
| Other; Other; Other | | | | |
| p_Firmicutes; c_Bacilli; Other; Other; Other; Other | 0.004%⁺ | 0.000% | Absent | Absent |
| p_Firmicutes; c_Clostridia; o_Clostridiales; f_{—} | 0.015%⁺ | 0.000% | Absent | Absent |
| [Mogibacteriaceae]; Other; Other | | | | |
| p_Firmicutes; c_Clostridia; o_Clostridiales; f_{—} | 0.010% | 0.000% | Absent | Absent |
| Clostridiaceae; g_02d06; s_{—} | | | | |
| p_Firmicutes; c_Clostridia; o_Clostridiales; f_{—} | 0.014% | 0.000% | Absent | Absent |
| Clostridiaceae; g_Clostridium; Other | | | | |
| p_Firmicutes; c_Clostridia; o_Clostridiales; f_{—} | 0.162%** | 0.000% | Absent | Absent |
| Clostridiaceae; g_SMB53; s_{—} | | | | |
| p_Firmicutes; c_Clostridia; o_Clostridiales; f_{—} | 0.119%** | 0.000% | Absent | Absent |
| Clostridiaceae; Other; Other | | | | |
| p_Firmicutes; c_Clostridia; o_Clostridiales; f_{—} | 0.004%⁺ | 0.000% | Absent | Absent |
| Lachnospiraceae; g_Blautia; s_producta | | | | |
| p_Firmicutes; c_Clostridia; o_Clostridiales; f_{—} | 0.003%⁺ | 0.000% | Absent | Absent |
| Lachnospiraceae; g_Coprococcus; s_catus | | | | |
| p_Firmicutes; c_Clostridia; o_Clostridiales; f_{—} | 0.004%⁺ | 0.000% | Absent | Absent |
| Lachnospiraceae; g_Moryella; s_indoligenes | | | | |
| p_Firmicutes; c_Clostridia; o_Clostridiales; f_{—} | 0.008%⁺ | 0.000% | Absent | Absent |
| Lachnospiraceae; g_Roseburia; Other | | | | |
| p_Firmicutes; c_Clostridia; o_Clostridiales; f_{—} | 0.060%⁺ | 0.000% | Absent | Absent |
| Lachnospiraceae; g_Roseburia; s_{—} | | | | |
| p_Firmicutes; c_Clostridia; o_Clostridiales; f_{—} | 0.022%⁺ | 0.000% | Absent | Absent |
| Peptococcaceae; g_rc4-4; s_{—} | | | | |
| p_Firmicutes; c_Clostridia; o_Clostridiales; f_{—} | 0.380%** | 0.000% | Absent | Absent |
| Peptostreptococcaceae; g_; s_{—} | | | | |
| p_Firmicutes; c_Clostridia; o_Clostridiales; f_{—} | 0.006%⁺ | 0.000% | Absent | Absent |
| Peptostreptococcaceae; Other; Other | | | | |
| p_Firmicutes; c_Clostridia; o_Clostridiales; f_{—} | 0.003%⁺ | 0.000% | Absent | Absent |
| Ruminococcaceae; g_Faecalibacterium; s_{—} | | | | |
| p_Firmicutes; c_Clostridia; Other; Other; Other; Other | 0.015%⁺ | 0.000% | Absent | Absent |
| p_Proteobacteria; c_Betaproteobacteria; o_Burkholderiales; | 0.004%⁺ | 0.000% | Absent | Absent |
| f_Comamonadaceae; g_Comamonas; s_{—} | | | | |
| p_Proteobacteria; c_Betaproteobacteria; | 0.007%⁺ | 0.000% | Absent | Absent |
| Other; Other; Other; Other | | | | |
| p_Proteobacteria; c_Deltaproteobacteria; o_{—} | 0.013%⁺ | 0.000% | Absent | Absent |
| Desulfovibrionales; Other; Other; Other | | | | |
| p_Tenericutes; c_Mollicutes; o_Anaeroplasmatales; | 0.425%** | 0.000% | Absent | Absent |
| f_Anaeroplasmataceae; g_Anaeroplasma; s_{—} | | | | |
| p_Tenericutes; c_Mollicutes; o_RF39; f_; g_; s_{—} | 0.146% | 0.000% | Absent | Absent |
| p_Firmicutes; c_Clostridia; o_Clostridiales; f_{—} | 4.148%* | 0.006%⁺ | 0.001 | −9.387 |
| Clostridiaceae; g_; s_{—} | | | | |
| p_Firmicutes; c_Bacilli; o_Lactobacillales; | 1.321%* | 0.003%⁺ | 0.002 | −8.736 |
| f_Lactobacillaceae; g_Lactobacillus; s_{—} | | | | |
| p_Firmicutes; c_Erysipelotrichi; o_Erysipelotrichales; | 26.062%* | 0.106%** | 0.004 | −7.946 |
| f_Erysipelotrichaceae; g_Allobaculum; s_{—} | | | | |
| p_Firmicutes; c_Bacilli; o_Turicibacterales; f_{—} | 0.726%** | 0.011%⁺ | 0.016 | −6.006 |
| Turicibacteraceae; g_Turicibacter; s_{—} | | | | |
| p_Bacteroidetes; c_Bacteroidia; o_Bacteroidales; | 0.418%** | 0.037%⁺ | 0.089 | −3.487 |
| f_Prevotellaceae; g_Prevotella; s_{—} | | | | |
| p_Firmicutes; c_Erysipelotrichi; o_Erysipelotrichales; | 0.036%⁺ | 0.003%⁺ | 0.097 | −3.368 |
| f_Erysipelotrichaceae; Other; Other | | | | |
| p_Firmicutes; c_Clostridia; o_Clostridiales; f_{—} | 0.121%** | 0.013%⁺ | 0.108 | −3.212 |
| Lachnospiraceae; g_Anaerostipes; s_{—} | | | | |
| p_Firmicutes; Other; Other; Other; Other; Other | 0.116% | 0.014%⁺ | 0.122 | −3.040 |
| p_Firmicutes; c_Bacilli; o_Lactobacillales; | 0.048%⁺ | 0.007%⁺ | 0.136 | −2.877 |
| f_Lactobacillaceae; g_Lactobacillus; Other | | | | |
| p_Proteobacteria; c_Betaproteobacteria; | 0.167%** | 0.029%⁺ | 0.175 | −2.515 |
| o_Burkholderiales; f_Alcaligenaceae; g_Sutterella; s_{—} | | | | |
| p_Firmicutes; c_Clostridia; o_Clostridiales; f_{—} | 0.057%⁺ | 0.014%⁺ | 0.252 | −1.991 |
| Ruminococcaceae; g_Anaerofilum; s_{—} | | | | |
| p_Firmicutes; c_Clostridia; o_Clostridiales; f_{—} | 0.014%⁺ | 0.004%⁺ | 0.264 | −1.921 |
| Ruminococcaceae; g_Ruminococcus; Other | | | | |
| p_Actinobacteria; c_Actinobacteria; o_Bifidobacteriales; | 0.157%** | 0.053%⁺ | 0.340 | −1.554 |
| f_Bifidobacteriaceae; g_Bifidobacterium; s_{—} | | | | |
| p_Firmicutes; c_Clostridia; o_Clostridiales; f_{—} | 1.657%* | 0.830%** | 0.501 | −0.998 |
| Lachnospiraceae; g_Dorea; s_{—} | | | | |
| p_Firmicutes; c_Clostridia; o_Clostridiales; f_{—} | 0.018%⁺ | 0.009%⁺ | 0.513 | −0.963 |
| Christensenellaceae; g_; s_{—} | | | | |
| p_Bacteroidetes; c_Bacteroidia; o_Bacteroidales; | 0.015%⁺ | 0.007%⁺ | 0.514 | −0.959 |
| f_Bacteroidaceae; g_Bacteroides; Other | | | | |
| p_Proteobacteria; c_Deltaproteobacteria; o_{—} | 0.026%⁺ | 0.015%⁺ | 0.587 | −0.769 |
| Desulfovibrionales; f_Desulfovibrionaceae; Other; Other | | | | |
| p_Firmicutes; c_Clostridia; o_Clostridiales; f_{—} | 0.010%⁺ | 0.007%⁺ | 0.668 | −0.582 |
| Lachnospiraceae; g_Butyrivibrio; s_{—} | | | | |
| p_Actinobacteria; c_Coriobacteriia; o_Coriobacteriales; | 0.015%⁺ | 0.011%⁺ | 0.739 | −0.437 |
| f_Coriobacteriaceae; g_Adlercreutzia; s_{—} | | | | |
| p_Firmicutes; c_Clostridia; o_Clostridiales; f_{—} | 0.358%** | 0.282%** | 0.786 | −0.347 |
| Lachnospiraceae; g_[Ruminococcus]; s_gnavus | | | | |
| p_Firmicutes; c_Bacilli; o_Lactobacillales; | 0.017%⁺ | 0.016%⁺ | 0.973 | −0.039 |
| f_Streptococcaceae; g_Lactococcus; s_{—} | | | | |
| p_Bacteroidetes; c_Bacteroidia; o_Bacteroidales; | 20.194%* | 19.801%* | 0.981 | −0.028 |
| f_S24-7; g_; s_{—} | | | | |
| p_Firmicutes; c_Clostridia; o_Clostridiales; f_{—} | 9.949%* | 9.925%* | 0.998 | −0.004 |
| Lachnospiraceae; g_; s_{—} | | | | |
| p_Bacteroidetes; c_Bacteroidia; o_Bacteroidales; | 5.158%* | 5.211%* | 1.010 | 0.015 |
| f_Rikenellaceae; g_; s_{—} | | | | |
| p_Firmicutes; c_Erysipelotrichi; o_Erysipelotrichales; | 0.006%⁺ | 0.007%⁺ | 1.019 | 0.027 |
| f_Erysipelotrichaceae; g_Coprobacillus; s_{—} | | | | |
| p_Firmicutes; c_Bacilli; o_Lactobacillales; | 0.004%⁺ | 0.004%⁺ | 1.024 | 0.035 |
| f_Enterococcaceae; g_Enterococcus; s_{—} | | | | |
| p_Firmicutes; c_Clostridia; o_Clostridiales; f_{—} | 0.007%⁺ | 0.007%⁺ | 1.028 | 0.041 |
| Ruminococcaceae; g_Faecalibacterium; Other | | | | |
| p_Firmicutes; c_Clostridia; o_Clostridiales; f_{—} | 0.433%** | 0.446%** | 1.030 | 0.042 |
| Ruminococcaceae; g_; s_{—} | | | | |
| Other; Other; Other; Other; Other; Other | 0.098%⁺ | 0.102% | 1.047 | 0.066 |
| p_Firmicutes; c_Clostridia; o_Clostridiales; f_{—} | 0.122%** | 0.129%** | 1.061 | 0.086 |
| Ruminococcaceae; Other; Other | | | | |
| p_Firmicutes; c_Clostridia; o_Clostridiales; f_{—} | 0.656%** | 0.707%** | 1.078 | 0.108 |
| Ruminococcaceae; g_Ruminococcus; s_{—} | | | | |
| p_Proteobacteria; c_Deltaproteobacteria; o_{—} | 1.726%* | 2.061%* | 1.194 | 0.256 |
| Desulfovibrionales; f_Desulfovibrionaceae; | | | | |
| g_Desulfovibrio; s_C21_c20 | | | | |
| p_Proteobacteria; c_Deltaproteobacteria; o_{—} | 0.003%⁺ | 0.003%⁺ | 1.199 | 0.261 |
| Desulfovibrionales; f_Desulfovibrionaceae; | | | | |
| g_Desulfovibrio; Other | | | | |
| p_Bacteroidetes; Other; Other; Other; Other; Other | 0.037%⁺ | 0.045%⁺ | 1.215 | 0.281 |
| p_Firmicutes; c_Clostridia; o_Clostridiales; | 0.497%** | 0.637%** | 1.281 | 0.358 |
| Other; Other; Other | | | | |
| p_Firmicutes; c_Clostridia; o_Clostridiales; f_{—} | 0.117%** | 0.152%** | 1.299 | 0.377 |
| [Mogibacteriaceae]; g_; s_{—} | | | | |
| p_Firmicutes; c_Clostridia; o_Clostridiales; f_{—} | 0.026%⁺ | 0.037%⁺ | 1.400 | 0.485 |
| Lachnospiraceae; g_Coprococcus; Other | | | | |
| p_Firmicutes; c_Clostridia; o_Clostridiales; f_{—} | 2.289%* | 3.311%* | 1.447 | 0.533 |
| Lachnospiraceae; g_Coprococcus; s_{—} | | | | |
| p_Proteobacteria; c_Deltaproteobacteria; o_{—} | 1.443%* | 2.187%* | 1.516 | 0.600 |
| Desulfovibrionales; f_Desulfovibrionaceae; | | | | |
| g_Bilophila; s_{—} | | | | |
| p_Proteobacteria; Other; Other; Other; Other; Other | 0.017%⁺ | 0.026%⁺ | 1.522 | 0.606 |
| p_Firmicutes; c_Clostridia; o_Clostridiales; f_{—} | 0.009%⁺ | 0.013%⁺ | 1.523 | 0.607 |
| Lachnospiraceae; g_Dorea; Other | | | | |
| p_Firmicutes; c_Clostridia; o_Clostridiales; f_{—} | 3.592%* | 5.570%* | 1.551 | 0.633 |
| Ruminococcaceae; g_Oscillospira; s_{—} | | | | |
| p_Firmicutes; c_Clostridia; o_Clostridiales; f_{—} | 1.775%* | 3.243%* | 1.827 | 0.870 |
| Lachnospiraceae; Other; Other | | | | |
| p_Bacteroidetes; c_Bacteroidia; o_Bacteroidales; | 0.511%** | 0.943%** | 1.846 | 0.884 |
| f_Bacteroidaceae; g_Bacteroides; s_ovatus | | | | |
| p_Firmicutes; c_Clostridia; o_Clostridiales; f_;g_; s_{—} | 5.820%* | 13.229%* | 2.273 | 1.185 |
| p_Bacteroidetes; c_Bacteroidia; o_Bacteroidales; | 0.073%⁺ | 0.168%** | 2.299 | 1.201 |
| f_Bacteroidaceae; g_Bacteroides; s_acidifaciens | | | | |
| p_Firmicutes; c_Clostridia; o_Clostridiales; f_{—} | 0.081%⁺ | 0.190%** | 2.334 | 1.223 |
| Dehalobacteriaceae; g_Dehalobacterium; s_{—} | | | | |
| p_Firmicutes; c_Clostridia; o_Clostridiales; f_{—} | 0.003%⁺ | 0.007%⁺ | 2.655 | 1.409 |
| Ruminococcaceae; g_Oscillospira; s_guilliermondii | | | | |
| p_Bacteroidetes; c_Bacteroidia; o_Bacteroidales; | 0.101%** | 0.318%** | 3.141 | 1.651 |
| Other; Other; Other | | | | |
| p_Bacteroidetes; c_Bacteroidia; o_Bacteroidales; | 0.007%⁺ | 0.023%⁺ | 3.157 | 1.659 |
| f_Bacteroidaceae; g_Bacteroides; s_{—} | | | | |
| p_Firmicutes; c_Clostridia; o_Clostridiales; f_{—} | 0.004%⁺ | 0.017%⁺ | 4.651 | 2.218 |
| Lachnospiraceae; g_Blautia; Other | | | | |
| p_Firmicutes; c_Clostridia; o_Clostridiales; f_{—} | 0.005%⁺ | 0.027%⁺ | 5.304 | 2.407 |
| Ruminococcaceae; g_Oscillospira; Other | | | | |
| p_Bacteroidetes; c_Bacteroidia; o_Bacteroidales; | 0.912%** | 4.862%* | 5.332 | 2.415 |
| f_[Odoribacteraceae]; g_Odoribacter; s_{—} | | | | |
| p_Firmicutes; c_Clostridia; o_Clostridiales; f_{—} | 0.015%⁺ | 0.156%** | 10.708 | 3.421 |
| Peptococcaceae; g_; s_{—} | | | | |
| p_Firmicutes; c_Erysipelotrichi; o_Erysipelotrichales; | 0.157%** | 2.266%* | 14.472 | 3.855 |
| f_Erysipelotrichaceae; g_; s_{—} | | | | |
| p_Verrucomicrobia; c_Verrucomicrobiae; o_Verrucomicrobiales; | 0.929%** | 16.170%* | 17.400 | 4.121 |
| f_Verrucomicrobiaceae; g_{—} | | | | |
| Akkermansia; s_muciniphila | | | | |
| p_Proteobacteria; c_Alphaproteobacteria; o_{—} | 0.015%⁺ | 0.282%** | 18.990 | 4.247 |
| RF32; f_; g_; s_{—} | | | | |
| p_Bacteroidetes; c_Bacteroidia; | 0.003%⁺ | 2.794%* | 1080.729 | 10.078 |
| o_Bacteroidales; f_; g_; s_{—} | | | | |
| p_Firmicutes; c_Clostridia; o_Clostridiales; f_{—} | 0.000% | 0.061%⁺ | Present | Present |
| Clostridiaceae; g_Clostridium; s_{—} | | | | |
| p_Proteobacteria; c_Deltaproteobacteria; | 0.000% | 0.015%⁺ | Present | Present |
| Other; Other; Other; Other | | | | |
| p_Proteobacteria; c_Alphaproteobacteria; | 0.000% | 0.007%⁺ | Present | Present |
| Other; Other; Other; Other | | | | |
| p_Verrucomicrobia; c_Verrucomicrobiae; | 0.000% | 0.004%⁺ | Present | Present |
| o_Verrucomicrobiales; f_Verrucomicrobiaceae; g_{—} | | | | |
| Akkermansia; Other | | | | |
| p_Firmicutes; c_Clostridia; o_Clostridiales; f_{—} | 0.000% | 0.003%⁺ | Present | Present |
| Lachnospiraceae; g_Roseburia; s_faecis | | | | |

Code for the Control Inoculum:
*relative abundance > 1%;
**= relative abundance 0.1-1%:
⁺= >0 but <0.1%,
white = 0, not detected.
Taxa over-represented in STAT contribute to obesity; taxa over-represented in Control protect against obesity.

### REFERENCES

1. O'Hara, A. & Shanahan, F. The gut flora as a forgotten organ. EMBO Rep 7, 688 (2006).
2. van Nood, E., et al. Duodenal Infusion of Donor Feces for Recurrent Clostridium difficile. New England Journal of Medicine 368, 407-415 (2013).
3. Brandt, L. J. & Aroniadis, O. C. An overview of fecal microbiota transplantation: techniques, indications, and outcomes. Gastrointestinal Endoscopy, 1-10 (2013).
4. Greenblum, S., Turnbaugh, P. J. & Borenstein, E. Metagenomic systems biology of the human gut microbiome reveals topological shifts associated with obesity and inflammatory bowel disease. Proc Natl Acad Sci USA 109, 594-599 (2012).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1. A method for transfer of gastrointestinal microbiota from a donor subject to a recipient subject comprising the steps of:
(a) specimen collection, wherein a microbiota sample is recovered from the donor subject and, within 10 minutes of collection, is placed in an airtight collection container with or without an anaerobic transport medium, and sealed to avoid contact with oxygen in the air;
(b) specimen preparation, wherein the microbiota sample collected in step (a) is prepared in an anaerobic environment, comprising (i) adding a reduced (no oxygen) sterile solution if the microbiota sample was not collected in solution in step (a) or optionally adding a reduced (no oxygen) sterile solution if the microbiota sample was collected in solution in step (a), followed by (ii) homogenization, (iii) removal of solids, and (iv) transfer to a transport container that is under an anaerobic environment and has an airtight cap;
(c) transport of the microbiota sample prepared in step (b) to the delivery site in the recipient subject in the transport container;
(d) removal of the microbiota from the transport container into a delivery vehicle with minimal oxygen exposure, and
(e) direct transfer of the microbiota to the gastrointestinal tract of the recipient subject using the delivery vehicle, with minimal oxygen exposure.

2. The method of claim 1, wherein in step (a) the microbiota sample is recovered from the donor subject by recovery of feces immediately after defecation or by removal of cecal, ileal, or colonic luminal contents.

3. The method of claim 1, wherein in the collection step (a), the microbiota sample is placed in an airtight container within 1 minute of collection.

4. The method of claim 1, wherein the transport medium is step (a) is a reduced (no oxygen) sterile solution.

5. (canceled)

6. The method of claim 1, wherein the anaerobic environment in step (b) is composed of
(i) 90% nitrogen, 5% hydrogen, and 5% carbon dioxide, or
(ii) 95% nitrogen and 5% hydrogen, or
(iii) 100% nitrogen.

7-9. (canceled)

10. The method of claim 1, wherein step (a) and/or (b) is followed by freezing the microbiota sample and thawing said sample before the next step.

11-13. (canceled)

14. The method of claim 1, wherein step (c) is conducted at 18-25° C.

15. (canceled)

16. The method of claim 1, wherein step (d) is conducted without opening the transport container with the microbiota sample using a needle (≦16 gauge) and syringe to pierce the airtight cap and draw up a sufficient volume of the microbiota suspension.

17. The method of claim 1, wherein step (d) is conducted by transferring the microbiota suspension to the delivery vehicle within 3 minutes of opening the container with the microbiota sample.

18. (canceled)

19. The method of claim 16, wherein step (e) is accomplished by replacing the needle with a delivery vehicle that allows direct placement of the microbiota suspension in the gastrointestinal tract of the recipient subject.

20-27. (canceled)

28. A method for treating a disease in a subject in need thereof, wherein the disease is selected from the group consisting of Clostridium difficile associated diarrhea (CDI), inflammatory bowel disease (IBD), irritable bowel syndrome (IBS), idiopathic constipation, celiac disease, short stature, and growth retardation, said method comprising administering to the subject a therapeutically effective amount of a fecal microbiota transplant transferred in accordance with the method of claim 1.

29. A method of treating or preventing weight gain and adiposity in a subject comprising administering to the subject a therapeutically effective amount of a microbiota inoculum comprising bacteria from the order Mollicutes order RF39 and/or Lactobacillales.

30. The method of claim 29, wherein the microbiota inoculum comprises bacteria from one or more families selected from the group consisting of Coriobacteriaceae, Rikenellaceae, Clostridiaceae, Peptostreptococcaceae, and Lactobacillaceae.

31. A method of treating or preventing weight gain and adiposity in a subject comprising administering to the subject a therapeutically effective amount of a microbiota inoculum comprising bacteria from one or more genera selected from the group consisting of Allobaculum, Klebsiella, Ruminococcus, Dorea, Lactobacillus, Peptococcaceae genus rc4-4, Desulfovibrio, Clostridiaceae genus SMB53, Roseburia, and Oscillospira.

32. The method of claim 31, wherein the microbiota inoculum comprises bacteria from the species Lactobacillus reuteri.

33. A method of promoting and/or enhancing weight gain and/or height gain and/or fat accumulation in a subject in need thereof comprising administering to the subject a therapeutically effective amount of a microbiota inoculum comprising bacteria from one or more families selected from the group consisting of Verrucomicrobiaceae, Lachnospiraceae, Porphyromonadaceae, and Enterococcaceae.

34. The method of claim 33, wherein the microbiota inoculum comprises bacteria from one or more genera selected from the group consisting of Akkermansia, Odoribacter, Enterococcus, and Blautia.

35. The method of claim 34, wherein the microbiota inoculum comprises bacteria from the species Akkermansia muciniphila and/or Blautia producta.

36-37. (canceled)

38. A method for identifying individuals at risk for an increase in weight, height, and adiposity in a subject, said method comprising detecting in the gastrointestinal microbiota of the subject one or more bacterial taxa selected from the group consisting of family Verrucomicrobiaceae, family Lachnospiraceae, family Porphyromonadaceae, family Enterococcaceae, genus Akkermansia, genus Odoribacter, genus Enterococcus, genus Blautia, species Akkermansia muciniphila, and species Blautia producta.

39. A method for predicting a decrease in weight, height, and adiposity in a subject, said method comprising detecting in the gastrointestinal microbiota of the subject one or more bacterial taxa selected from the group consisting of order Mollicutes order RF39, order Lactobacillales, family Coriobacteriaceae, family Rikenellaceae, family Clostridiaceae, family Peptostreptococcaceae, family Lactobacillaceae, genus Allobaculum, genus Klebsiella, genus Ruminococcus, genus Dorea, genus Lactobacillus, genus Peptococcaceae genus rc4-4, genus Desulfovibrio, genus Clostridiaceae genus SMB53, genus Roseburia, genus Oscillospira, and species Lactobacillus reuteri.

40-41. (canceled)
